(19) United States
(12) Patent Application Publication
(10) Pub. No.: US 2026/0273048 A1
(43) Pub. Date: Sep. 17, 2026

(54) METHODS OF IMPROVING TRANSPLANT SURVIVAL USING IL-2 RECEPTOR GAMMA CHAIN ANTIBODIES

(71) Applicant: Regeneron Pharmaceuticals, Inc., Tarrytown, NY (US)

(72) Inventors: Audrey Le Floc'h, Hastings-on-Hudson, NY (US); Jamie M. Orengo, Cortlandt Manor, NY (US); George C. Scott, Glen Ridge, NJ (US)

(21) Appl. No.: 19/471,712

(22) PCT Filed: Apr. 1, 2024
(86) PCT No.: PCT/US2024/022447

§ 371 Date: Oct. 2, 2025

Related U.S. Application Data
(60) Provisional application No. 63/493,860, filed on Apr. 3, 2023.
(60) Provisional application No. 63/516,394, filed on Jul. 28, 2023.

(51) Int. Cl. A61K 39/395; A61K 31/5377; A61K 38/17; A61P 37/06

(52) U.S. Cl. CPC A61K 39/3955; A61K 31/5377; A61K 38/1774; A61P 37/06

(57) ABSTRACT

The present disclosure provides methods for improving transplant survival or delaying the onset of transplant rejection by administering to a subject an IL-2Rγ antibody or a combination therapy comprising an IL2Rγ antibody and one or more additional therapeutic agents, such a CD28 signaling inhibitor such as a CTLA4-Ig fusion protein.

### SEQUENCE LISTING

The sequence listing of the present application is submitted electronically as an ST.26 formatted xml file with a file name “SeqList-11425,” creation date of Feb. 20, 2024, and a size of 9,908 bytes. This sequence listing submitted is part of the specification and is hereby incorporated by reference in its entirety. The following sequence has a length that is below the minimum length permitted under ST.26 format: AAS (SEQ ID NO: 7).

### FIELD

The present disclosure relates to methods of improving transplant survival. The methods include administering to a subject in need thereof an IL-2Rγ antibody, or a combination therapy comprising an IL-2Rγ antibody and one or more additional immunosuppressive agents, such as a CTLA4-Ig fusion protein.

### BACKGROUND

Following organ or tissue transplantation, immunosuppressive therapy is utilized in an effort to prevent transplant rejection, improve transplant and patient survival, and reduce complications. Immunosuppressive therapy can generally be divided into three categories: induction, maintenance, and treatment of rejection. Induction typically involves the use of high-intensity immunosuppressive therapy at the time of or immediately after the transplant. Induction therapy most commonly involves the use of T-cell depleting agents, such as anti-thymocyte globulin (ATG) and alemtuzumab. Although induction therapy can lower the incidence of acute rejection episodes, the therapies are associated with significant adverse effects. In particular, the use of T-cell depleting agents is associated with higher rates of infection, neutropenia, thrombocytopenia, and cytokine-release syndrome (Enderby and Keller, Am J Manag Care 2015, 21:S12S-23).

After the induction phase, long-term maintenance immunosuppressive therapy is required. The long-term maintenance of immunosuppression following transplantation is complex, and typically involves the use of a combination of agents which can vary based on various factors including donor, recipient, and transplant characteristics (Pilch et al., Pharmacotherapy 2021, 41:119-131). Commonly used maintenance immunosuppressive agents include calcineurin inhibitors, antiproliferatives/antimetabolites, corticosteroids, mammalian target of rapamycin (mTOR) inhibitors, and T-cell costimulation blockers (Enderby and Keller, Am J Manag Care 2015, 21:S12S-23). One such T-cell costimulation blocker is belatacept, a high-affinity CTLA4-Ig fusion protein that is approved in the US for use to prevent kidney transplant rejection in combination with mycophenolate and corticosteroids. To date, CTLA4-Ig fusion proteins have not been shown to be effective as a monotherapy in inducing long-term graft acceptance (Iglesias et al., Front. Immunol. 2022, 13:926648). Additionally, there are concerns about the possible impact on Treg homeostasis from long-term administration of CTLA-Ig (Riella et al., Am J Transplant 2012, 12:846-855). Thus, there remains a significant need for agents that prevent transplant rejection and improve transplant survival.

### SUMMARY

In one aspect, the present disclosure provides methods of improving organ or tissue transplant survival. In some embodiments, the method comprises administering to a subject at least one dose of an antibody that specifically binds to interleukin 2 receptor gamma chain (IL-2Rγ), or an antigen-binding fragment thereof, prior to an organ or tissue transplant in the subject. In some embodiments, the method comprises administering to a subject at least one dose of an antibody that specifically binds to IL-2Rγ, or an antigen-binding fragment thereof, prior to an organ or tissue transplant in the subject; and further administering to the subject one or more doses of a maintenance immunosuppressive therapy, such as an antiproliferative agent or a T cell costimulatory pathway blocker.

In some embodiments, the method comprises: administering to a subject at least one dose of an antibody that specifically binds to IL-2Rγ, or an antigen-binding fragment thereof, prior to an organ or tissue transplant in the subject; and administering to the subject one or more doses of a CD28 signaling inhibitor.

In some embodiments, the method comprises: administering to a subject at least one dose of an antibody that specifically binds to IL-2Rγ, or an antigen-binding fragment thereof, prior to an organ or tissue transplant in the subject; and administering to the subject one or more doses of an antiproliferative agent.

In some embodiments, the organ or tissue transplant is a bone transplant, corneal transplant, heart transplant, heart valve transplant, kidney transplant, ligament transplant, liver transplant, lung transplant, pancreas transplant, skin transplant, tendon transplant, trachea transplant, or vascular tissue transplant.

In some embodiments, the IL-2Rγ antibody or antigen-binding fragment thereof comprises three HCDRs (HCDR1, HCDR2 and HCDR3) and three LCDRs (LCDR1, LCDR2 and LCDR3), wherein the HCDR1 comprises the amino acid sequence of SEQ ID NO:2, the HCDR2 comprises the amino acid sequence of SEQ ID NO:3, the HCDR3 comprises the amino acid sequence of SEQ ID NO:4, the LCDR1 comprises the amino acid sequence of SEQ ID NO:6, the LCDR2 comprises the amino acid sequence AAS, and the LCDR3 comprises the amino acid sequence of SEQ ID NO:8. In some embodiments, the IL-2Rγ antibody or antigen-binding fragment thereof comprises a heavy chain variable region (HCVR) comprising the amino acid sequence of SEQ ID NO: 1 and a light chain variable region (LCVR) comprising the amino acid sequence of SEQ ID NO:5. In some embodiments, the IL-2Rγ antibody or antigen-binding fragment thereof comprises a heavy chain comprising the amino acid sequence of SEQ ID NO:9 and a light chain comprising the amino acid sequence of SEQ ID NO:10.

In some embodiments, the method comprises administering to the subject two or more doses of the IL-2Rγ antibody or antigen-binding fragment thereof prior to the organ or tissue transplant. In some embodiments, the method comprises administering to the subject one or more doses of the IL-2Rγ antibody or antigen-binding fragment thereof prior to the organ or tissue transplant and one or more doses of the IL-2Rγ antibody or antigen-binding fragment thereof after the organ or tissue transplant. In some embodiments, the IL-2Rγ antibody or antigen-binding fragment thereof is administered to the subject once a week, two times a week, or three times a week.

In some embodiments, the CD28 signaling inhibitor is a fusion protein comprising an extracellular portion of cytotoxic T lymphocyte-associated protein 4 (CTLA4) and an Fc region of immunoglobulin IgG1 (Ig). In some embodiments, the CTLA4-Ig fusion protein is abatacept or belatacept. In some embodiments, the CD28 signaling inhibitor is administered to the subject starting on the day of the organ or tissue transplant. In some embodiments, at least one dose of the CD28 signaling inhibitor is administered to the subject prior to the organ or tissue transplant. In some embodiments, the CD28 signaling inhibitor is administered to the subject three times a week, two times a week, once a week, or once every two weeks.

In some embodiments, the method further comprises administering to the subject a CD40L inhibitor. In some embodiments, the CD40L inhibitor is a CD40L antibody or an antigen-binding fragment thereof, or a fusion protein that specifically binds to CD40L. In some embodiments, the CD40L inhibitor is dapirolizumab pegol, dazodalibep, frexalimab, letolizumab, ruplizumab, tegoprubart, or MR-1. In some embodiments, the CD40L inhibitor is administered to the subject once a week, two times a week, or three times a week.

In some embodiments, (a) the CD28 signaling inhibitor is abatacept and the CD40L inhibitor is dapirolizumab pegol, dazodalibep, frexalimab, letolizumab, ruplizumab, tegoprubart, or MR-1; or (b) the CD28 signaling inhibitor is belatacept and the CD40L inhibitor is dapirolizumab pegol, dazodalibep, frexalimab, letolizumab, ruplizumab, tegoprubart, or MR-1; or (c) the CD28 signaling inhibitor is abatacept and the CD40L inhibitor is MR-1; or (d) the CD28 signaling inhibitor is belatacept and the CD40L inhibitor is MR-1.

In some embodiments, one or more of the IL-2Rγ antibody or antigen-binding fragment thereof, the CD28 signaling inhibitor, and the CD40L inhibitor are administered to the subject starting at least two days prior to the organ or tissue transplant. In some embodiments, one or more of the IL-2Rγ antibody or antigen-binding fragment thereof, the CD28 signaling inhibitor, and the CD40L inhibitor are administered to the subject until the onset of transplant rejection.

In some embodiments, the antiproliferative agent is mycophenolate (e.g., mycophenolate mofetil (MMF) or mycophenolate sodium), azathioprine (AZA), or leflunomide. In some embodiments, the antiproliferative agent is MMF. In some embodiments, the antiproliferative agent is administered to the subject starting on the day of the organ or tissue transplant. In some embodiments, at least one dose of the antiproliferative agent is administered to the subject prior to the organ or tissue transplant. In some embodiments, the antiproliferative agent is administered to the subject daily, six times a week, five times a week, four times a week, three times a week, two times a week, once a week, or once every two weeks.

In another aspect, the present disclosure provides methods for delaying the onset of transplant rejection in a subject. In some embodiments, the method comprises administering to a subject at least one dose of an antibody that specifically binds to IL-2Rγ, or an antigen-binding fragment thereof, prior to an organ or tissue transplant in the subject. In some embodiments, the method comprises administering to a subject at least one dose of an antibody that specifically binds to IL-2Rγ, or an antigen-binding fragment thereof, prior to an organ or tissue transplant in the subject; and further administering to the subject one or more doses of a maintenance immunosuppressive therapy, such as an antiproliferative agent or a T cell costimulatory pathway blocker.

In some embodiments, the method comprises: administering to a subject at least one dose of an antibody that specifically binds to IL-2Rγ, or an antigen-binding fragment thereof, prior to an organ or tissue transplant in the subject; and administering to the subject one or more doses of a CD28 signaling inhibitor.

In some embodiments, the method comprises: administering to a subject at least one dose of an antibody that specifically binds to IL-2Rγ, or an antigen-binding fragment thereof, prior to an organ or tissue transplant in the subject; and administering to the subject one or more doses of an antiproliferative agent.

In some embodiments, the organ or tissue transplant is a bone transplant, corneal transplant, heart transplant, heart valve transplant, kidney transplant, ligament transplant, liver transplant, lung transplant, pancreas transplant, skin transplant, tendon transplant, trachea transplant, or vascular tissue transplant.

In some embodiments, the IL-2Rγ antibody or antigen-binding fragment thereof comprises three HCDRs (HCDR1, HCDR2 and HCDR3) and three LCDRs (LCDR1, LCDR2 and LCDR3), wherein the HCDR1 comprises the amino acid sequence of SEQ ID NO:2, the HCDR2 comprises the amino acid sequence of SEQ ID NO:3, the HCDR3 comprises the amino acid sequence of SEQ ID NO:4, the LCDR1 comprises the amino acid sequence of SEQ ID NO:6, the LCDR2 comprises the amino acid sequence AAS, and the LCDR3 comprises the amino acid sequence of SEQ ID NO:8. In some embodiments, the IL-2Rγ antibody or antigen-binding fragment thereof comprises an HCVR comprising the amino acid sequence of SEQ ID NO:1 and an LCVR comprising the amino acid sequence of SEQ ID NO:5. In some embodiments, the IL-2Rγ antibody or antigen-binding fragment thereof comprises a heavy chain comprising the amino acid sequence of SEQ ID NO:9 and a light chain comprising the amino acid sequence of SEQ ID NO: 10.

In some embodiments, the method comprises administering to the subject two or more doses of the IL-2Rγ antibody or antigen-binding fragment thereof prior to the organ or tissue transplant. In some embodiments, the method comprises administering to the subject one or more doses of the IL-2Rγ antibody or antigen-binding fragment thereof prior to the organ or tissue transplant and one or more doses of the IL-2Rγ antibody or antigen-binding fragment thereof after the organ or tissue transplant. In some embodiments, the IL-2Rγ antibody or antigen-binding fragment thereof is administered to the subject once a week, two times a week, or three times a week.

In some embodiments, the CD28 signaling inhibitor is an CTLA4-Ig fusion protein. In some embodiments, the CTLA4-Ig fusion protein is abatacept or belatacept. In some embodiments, the CD28 signaling inhibitor is administered to the subject starting on the day of the organ or tissue transplant. In some embodiments, at least one dose of the CD28 signaling inhibitor is administered to the subject prior to the organ or tissue transplant. In some embodiments, the CD28 signaling inhibitor is administered to the subject three times a week, two times a week, once a week, or once every two weeks.

In some embodiments, the method further comprises administering to the subject a CD40L inhibitor. In some embodiments, the CD40L inhibitor is a CD40L antibody or an antigen-binding fragment thereof, or a fusion protein that specifically binds to CD40L. In some embodiments, the CD40L inhibitor is dapirolizumab pegol, dazodalibep, frexalimab, letolizumab, ruplizumab, tegoprubart, or MR-1. In some embodiments, the CD40L inhibitor is administered to the subject once a week, two times a week, or three times a week.

In some embodiments, (a) the CD28 signaling inhibitor is abatacept and the CD40L inhibitor is dapirolizumab pegol, dazodalibep, frexalimab, letolizumab, ruplizumab, tegoprubart, or MR-1; or (b) the CD28 signaling inhibitor is belatacept and the CD40L inhibitor is dapirolizumab pegol, dazodalibep, frexalimab, letolizumab, ruplizumab, tegoprubart, or MR-1; or (c) the CD28 signaling inhibitor is abatacept and the CD40L inhibitor is MR-1; or (d) the CD28 signaling inhibitor is belatacept and the CD40L inhibitor is MR-1.

In some embodiments, one or more of the IL-2Rγ antibody or antigen-binding fragment thereof, the CD28 signaling inhibitor, and the CD40L inhibitor are administered to the subject starting at least two days prior to the organ or tissue transplant. In some embodiments, one or more of the IL-2Rγ antibody or antigen-binding fragment thereof, the CD28 signaling inhibitor, and the CD40L inhibitor are administered to the subject until the onset of transplant rejection.

In some embodiments, the antiproliferative agent is mycophenolate (e.g., MMF or mycophenolate sodium), AZA, or leflunomide. In some embodiments, the antiproliferative agent is MMF. In some embodiments, the antiproliferative agent is administered to the subject starting on the day of the organ or tissue transplant. In some embodiments, at least one dose of the antiproliferative agent is administered to the subject prior to the organ or tissue transplant. In some embodiments, the antiproliferative agent is administered to the subject daily, six times a week, five times a week, four times a week, three times a week, two times a week, once a week, or once every two weeks.

In another aspect, the present disclosure provides methods of improving organ or tissue transplant survival or delaying the onset of transplant rejection in a subject who is taking or has been prescribed maintenance immunosuppressive therapy, such as an antiproliferative agent or a T cell costimulatory pathway blocker. In some embodiments, the method comprises: administering to a subject at least one dose of an antibody that specifically binds to IL-2Rγ, or an antigen-binding fragment thereof, prior to an organ or tissue transplant in the subject; wherein the subject is taking or has been prescribed a CD28 signaling inhibitor. In some embodiments, the method comprises: administering to a subject at least one dose of an antibody that specifically binds to IL-2Rγ, or an antigen-binding fragment thereof, prior to an organ or tissue transplant in the subject; wherein the subject is taking or has been prescribed an antiproliferative agent.

In some embodiments, the organ or tissue transplant is a bone transplant, corneal transplant, heart transplant, heart valve transplant, kidney transplant, ligament transplant, liver transplant, lung transplant, pancreas transplant, skin transplant, tendon transplant, trachea transplant, or vascular tissue transplant.

In some embodiments, the CD28 signaling inhibitor a CTLA4-Ig fusion protein. In some embodiments, the CTLA4-Ig fusion protein is abatacept or belatacept. In some embodiments, the subject is taking the CD28 signaling inhibitor at least one day before the organ or tissue transplant. In some embodiments, the subject is taking the CD28 signaling inhibitor starting on the day of the organ or tissue transplant.

In some embodiments, the antiproliferative agent is mycophenolate (e.g., MMF or mycophenolate sodium), AZA, or leflunomide. In some embodiments, the antiproliferative agent is MMF. In some embodiments, the subject is taking the antiproliferative agent at least one day before the organ or tissue transplant. In some embodiments, the subject is taking the antiproliferative agent starting on the day of the organ or tissue transplant.

In some embodiments, the IL-2Rγ antibody or antigen-binding fragment thereof comprises three HCDRs (HCDR1, HCDR2 and HCDR3) and three LCDRs (LCDR1, LCDR2 and LCDR3), wherein the HCDR1 comprises the amino acid sequence of SEQ ID NO:2, the HCDR2 comprises the amino acid sequence of SEQ ID NO:3, the HCDR3 comprises the amino acid sequence of SEQ ID NO:4, the LCDR1 comprises the amino acid sequence of SEQ ID NO:6, the LCDR2 comprises the amino acid sequence AAS, and the LCDR3 comprises the amino acid sequence of SEQ ID NO:8. In some embodiments, the IL-2Rγ antibody or antigen-binding fragment thereof comprises an HCVR comprising the amino acid sequence of SEQ ID NO:1 and an LCVR comprising the amino acid sequence of SEQ ID NO:5. In some embodiments, the IL-2Rγ antibody or antigen-binding fragment thereof comprises a heavy chain comprising the amino acid sequence of SEQ ID NO:9 and a light chain comprising the amino acid sequence of SEQ ID NO: 10.

In some embodiments, the method comprises administering to the subject: two or more doses of the IL-2Rγ antibody or antigen-binding fragment thereof prior to the organ or tissue transplant; or one or more doses of the IL-2Rγ antibody or antigen-binding fragment thereof prior to the organ or tissue transplant and one or more doses of the IL-2Rγ antibody or antigen-binding fragment thereof after the organ or tissue transplant.

In some embodiments, the IL-2Rγ antibody or antigen-binding fragment thereof is administered to the subject once a week, two times a week, or three times a week.

In another aspect, the present disclosure provides combinations for use in improving organ or tissue transplant survival or for use in delaying the onset of transplant rejection in a subject. In some embodiments, the combination comprises: an antibody that specifically binds to IL-2Rγ or an antigen-binding fragment thereof; and a CD28 signaling inhibitor.

In some embodiments, the organ or tissue transplant is a bone transplant, corneal transplant, heart transplant, heart valve transplant, kidney transplant, ligament transplant, liver transplant, lung transplant, pancreas transplant, skin transplant, tendon transplant, trachea transplant, or vascular tissue transplant.

In some embodiments, the IL-2Rγ antibody or antigen-binding fragment thereof comprises three HCDRs (HCDR1, HCDR2 and HCDR3) and three LCDRs (LCDR1, LCDR2 and LCDR3), wherein the HCDR1 comprises the amino acid sequence of SEQ ID NO:2, the HCDR2 comprises the amino acid sequence of SEQ ID NO:3, the HCDR3 comprises the amino acid sequence of SEQ ID NO:4, the LCDR1 comprises the amino acid sequence of SEQ ID NO:6, the LCDR2 comprises the amino acid sequence AAS, and the LCDR3 comprises the amino acid sequence of SEQ ID NO:8. In some embodiments, the IL-2Rγ antibody or antigen-binding fragment thereof comprises an HCVR comprising the amino acid sequence of SEQ ID NO: 1 and an LCVR comprising the amino acid sequence of SEQ ID NO:5. In some embodiments, the IL-2Rγ antibody or antigen-binding fragment thereof comprises a heavy chain comprising the amino acid sequence of SEQ ID NO:9 and a light chain comprising the amino acid sequence of SEQ ID NO: 10.

In some embodiments, the CD28 signaling inhibitor is a CTLA4-Ig fusion protein. In some embodiments, the CTLA4-Ig fusion protein is abatacept or belatacept.

In some embodiments, the combination further comprises a CD40L inhibitor. In some embodiments, the CD40L inhibitor is a CD40L antibody or an antigen-binding fragment thereof, or a fusion protein that specifically binds to CD40L. In some embodiments, the CD40L inhibitor is dapirolizumab pegol, dazodalibep, frexalimab, letolizumab, ruplizumab, tegoprubart, or MR-1.

In some embodiments, the combination comprises: an antibody that specifically binds to IL-2Rγ or an antigen-binding fragment thereof; and an antiproliferative agent.

In some embodiments, the antiproliferative agent is mycophenolate (e.g., MMF or mycophenolate sodium), AZA, or leflunomide. In some embodiments, the antiproliferative agent is MMF.

In another aspect, the present disclosure provides an IL-2Rγ antibody or antigen-binding fragment thereof, or an IL-2Rγ antibody or antigen-binding fragment thereof and a CD28 signaling inhibitor, for use in the preparation of a medicament for improving organ or tissue transplant survival, or for delaying the onset of transplant rejection in a subject. In some embodiments, the subject is taking or has been prescribed a CD28 signaling inhibitor.

In some embodiments, IL-2Rγ antibody or antigen-binding fragment thereof comprises three HCDRs (HCDR1, HCDR2 and HCDR3) and three LCDRs (LCDR1, LCDR2 and LCDR3), wherein the HCDR1 comprises the amino acid sequence of SEQ ID NO:2, the HCDR2 comprises the amino acid sequence of SEQ ID NO:3, the HCDR3 comprises the amino acid sequence of SEQ ID NO:4, the LCDR1 comprises the amino acid sequence of SEQ ID NO:6, the LCDR2 comprises the amino acid sequence AAS, and the LCDR3 comprises the amino acid sequence of SEQ ID NO:8. In some embodiments, the IL-2Rγ antibody or antigen-binding fragment thereof comprises an HCVR comprising the amino acid sequence of SEQ ID NO: 1 and an LCVR comprising the amino acid sequence of SEQ ID NO:5. In some embodiments, the IL-2Rγ antibody or antigen-binding fragment thereof comprises a heavy chain comprising the amino acid sequence of SEQ ID NO:9 and a light chain comprising the amino acid sequence of SEQ ID NO: 10.

In some embodiments, the CD28 signaling inhibitor is a CTLA4-Ig fusion protein. In some embodiments, the CTLA4-Ig fusion protein is abatacept or belatacept.

In another aspect, the present disclosure provides an IL-2Rγ antibody or antigen-binding fragment thereof, or an IL-2Rγ antibody or antigen-binding fragment thereof and an antiproliferative agent, for use in the preparation of a medicament for improving organ or tissue transplant survival, or for delaying the onset of transplant rejection in a subject. In some embodiments, the subject is taking or has been prescribed an antiproliferative agent. In some embodiments, the antiproliferative agent is mycophenolate (e.g., MMF or mycophenolate sodium), AZA, or leflunomide. In some embodiments, the antiproliferative agent is MMF.

Other embodiments will be apparent from a review of the ensuing detailed description.

### BRIEF DESCRIPTION OF THE FIGURES

FIG. 1 shows the experimental layout for the in vivo skin graft rejection experiment disclosed in Example 1.

FIG. 2A shows the number of days post-surgery to the onset of skin graft rejection for mice without antibody treatment, mice treated with isotype control, or mice treated with the IL-2Rγ antibody REGN7257.

FIG. 2B shows the number of days post-surgery to complete skin graft rejection for mice without antibody treatment, mice treated with isotype control, or mice treated with the IL-2Rγ antibody REGN7257.

FIG. 3 shows total IgG donor-specific antibody levels in blood sampled at the day 56 post-transplant timepoint from mice without antibody treatment, mice treated with isotype control, mice treated with the IL-2Rγ antibody REGN7257, and non-engrafted control mice.

FIG. 4 shows the experimental layout for the in vivo skin graft rejection experiment disclosed in Example 2.

FIG. 5 shows skin graft survival and skin graft median survival time for mice without antibody treatment, mice treated with isotype induction and abatacept maintenance, mice treated with REGN7257 induction and isotype maintenance, mice treated with isotype induction and isotype maintenance, or mice treated with REGN7257 induction and abatacept maintenance.

FIG. 6 shows the experimental layout for the in vivo skin graft rejection experiment disclosed in Example 3.

FIG. 7 shows skin graft survival and skin graft median survival time for mice without antibody treatment, mice treated with isotype control, mice treated with abatacept and REGN7257, mice treated with CD40L antibody, mice treated with abatacept and CD40L antibody, and mice treated with REGN7257, abatacept, and CD40L antibody.

FIG. 8 shows skin graft survival and skin graft median survival time for mice without antibody treatment, mice treated with isotype control, mice treated with REGN7257 and abatacept, mice treated with REGN7257 and CD40L antibody, mice treated with abatacept and CD40L antibody, and mice treated with REGN7257, abatacept, and CD40L antibody.

FIG. 9 shows skin graft survival and skin graft median survival time for mice without antibody treatment, mice treated with isotype control, mice treated with mycophenolate mofetil (MMF), mice treated with REGN7257, and mice treated with REGN7257 and MMF.

### DETAILED DESCRIPTION

### Definitions

It is to be understood that the present disclosure is not limited to particular methods and experimental conditions described, as such methods and conditions may vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the present disclosure, preferred methods and materials are now described. All publications mentioned herein are hereby incorporated by reference in their entirety unless otherwise stated.

To aid in understanding the detailed description of the compositions and methods according to the disclosure, a few express definitions are provided to facilitate an unambiguous disclosure of the various aspects of the disclosure.

As used herein, the singular forms “a,” “an,” and “the” include plural references unless the context clearly dictates otherwise.

As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

The terms “IL-2 receptor gamma chain”, “IL-2Rγ,” “IL-2Rg,” and “IL-2RγC” interchangeably refer to a cytokine receptor subunit that forms part of the IL-2 receptor complex. IL-2Rγ was initially identified as the third subunit of the IL-2 receptor complex, but has also been identified as a subunit of other cytokine receptor complexes (IL-4R, IL-7R, IL-9R, IL-15R, and IL-21R complexes) and is also referred to as “common cytokine receptor gamma chain.” In some embodiments, the IL-2Rγ is human IL-2Rγ. In some embodiments, human IL-2Rγ is encoded by the nucleotide sequence set forth under NCBI Accession No. NM_000206. In some embodiments, human IL-2Rγ comprises the amino acid sequence set forth under NCBI Accession No. NP_000197.

The term “antibody,” as used herein, refers to an antigen-binding molecule or molecular complex comprising a set of complementarity determining regions (CDRs) that specifically bind to or interact with a particular antigen (e.g., IL-2Rγ). The term “antibody,” as used herein, includes immunoglobulin molecules comprising four polypeptide chains, two heavy (H) chains and two light (L) chains inter-connected by disulfide bonds, as well as multimers thereof (e.g., IgM). In a typical antibody, each heavy chain comprises a heavy chain variable region (abbreviated herein as HCVR or V_{H}) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In some embodiments, the FRs of the antibody (or antigen-binding portion thereof) may be identical to the human germline sequences, or may be naturally or artificially modified. An amino acid consensus sequence may be defined based on a side-by-side analysis of two or more CDRs.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add, or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

The term “antibody,” as used herein, also includes multispecific (e.g., bispecific) antibodies. A multispecific antibody or antigen-binding fragment of an antibody will typically comprise at least two different variable domains, wherein each variable domain is capable of specifically binding to a separate antigen or to a different epitope on the same antigen. Any multispecific antibody format may be adapted for use in the context of an antibody or antigen-binding fragment of an antibody of the present disclosure using routine techniques available in the art. Exemplary bispecific formats that can be used in the context of the present disclosure include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED) body, leucine zipper, Duobody, IgG1/IgG2, dual acting Fab (DAF)-IgG, and Mab² bispecific formats (see, e.g., Klein et al. 2012, mAbs 4:6, 1-11, and references cited therein, for a review of the foregoing formats). Bispecific antibodies can also be constructed using peptide/nucleic acid conjugation, e.g., wherein unnatural amino acids with orthogonal chemical reactivity are used to generate site-specific antibody-oligonucleotide conjugates which then self-assemble into multimeric complexes with defined composition, valency and geometry. (See, e.g., Kazane et al., J. Am. Chem. Soc. [Epub: Dec. 4, 2012]).

The term “human antibody,” as used herein, is intended to include antibodies having variable and constant regions derived from human germline immunoglobulin sequences. The human antibodies of the disclosure may nonetheless include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include antibodies in which CDR sequences derived from the germline of another mammalian species, such as a mouse, have been grafted onto human framework sequences.

The term “recombinant human antibody,” as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see, e.g., Taylor et al. (1992) Nucl. Acids Res. 20:6287-6295) or antibodies prepared, expressed, created, or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

An “isolated antibody” refers to an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody.” An isolated antibody also includes an antibody in situ within a recombinant cell. Isolated antibodies are antibodies that have been subjected to at least one purification or isolation step. According to certain embodiments, an isolated antibody may be substantially free of other cellular material and/or chemicals.

The term “specifically binds,” or the like, means that an antibody or antigen-binding fragment thereof forms a complex with an antigen that is relatively stable under physiologic conditions. Specific binding can be characterized by an equilibrium dissociation constant of at least about 1×10⁻⁶ M or less (e.g., a smaller K_{D} denotes a tighter binding). Methods for determining whether an antibody specifically binds to an antigen are well known in the art and include, for example, equilibrium dialysis, surface plasmon resonance, and the like. In some embodiments, specific binding is measured in a surface plasmon resonance assay. An isolated antibody that specifically binds an antigen from one species may or may not have cross-reactivity to other antigens, such as an orthologous antigen from another species.

The term “K_{D},” as used herein, refers to the equilibrium dissociation constant of a particular antibody-antigen interaction.

The term “surface plasmon resonance,” as used herein, refers to an optical phenomenon that allows for the analysis of real-time biomolecular interactions by detection of alterations in protein concentrations within a biosensor matrix, for example using the BIACORE™ system (Pharmacia Biosensor AB, Uppsala, Sweden and Piscataway, N.J.).

The terms “substantial identity” and “substantially identical,” as used with reference to a nucleic acid or fragment thereof, indicates that, when optimally aligned with appropriate nucleotide insertions or deletions with another nucleic acid (or its complementary strand), there is nucleotide sequence identity in at least about 90%, e.g., at least 91%, 92%, 93%, 94%, 95%, 96%, 97%, 98% or 99%, of the nucleotide bases, as measured by any well-known algorithm of sequence identity, such as FASTA, BLAST or GAP, as discussed below. A nucleic acid molecule having substantial identity to a reference nucleic acid molecule may, in certain instances, encode a polypeptide having the same or substantially similar amino acid sequence as the polypeptide encoded by the reference nucleic acid molecule.

As applied to polypeptides, the terms “substantial identity” and “substantially identical” mean that two peptide sequences, when optimally aligned, share at least about 90% sequence identity, e.g., at least 90%, 91%, 92%, 93%, 94%, 95%, 96%, 97%, 98% or 99% sequence identity. In some embodiments, residue positions that are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein.

Sequence similarity for polypeptides is typically measured using sequence analysis software. Protein analysis software matches similar sequences using measures of similarity assigned to various substitutions, deletions and other modifications, including conservative amino acid substitutions. For instance, GCG software contains programs such as GAP and BESTFIT which can be used with default parameters to determine sequence homology or sequence identity between closely related polypeptides, such as homologous polypeptides from different species of organisms or between a wild-type protein and a mutein thereof. See, e.g., GCG Version 6.1. Polypeptide sequences also can be compared using FASTA with default or recommended parameters; a program in GCG Version 6.1. FASTA (e.g., FASTA2 and FASTA3) provides alignments and percent sequence identity of the regions of the best overlap between the query and search sequences (Pearson, 2000 supra). Another preferred algorithm when comparing a sequence of the present disclosure to a database containing a large number of sequences from different organisms is the computer program BLAST, especially BLASTP or TBLASTN, using default parameters. (See, e.g., Altschul et al., 1990, J. Mol. Biol. 215:403-410 and 1997 Nucleic Acids Res. 25:3389-3402).

As used herein, the term “subject” refers to a human or non-human animal in need of amelioration, prevention and/or treatment of a disease or disorder (such as prevention of transplant rejection). In some embodiments, the subject is a human or non-human animal who has received, or will be receiving, an organ or tissue transplant. The terms “subject” and “patient” are used interchangeably herein.

As used herein, the terms “treat,” “treating,” or the like, mean to alleviate symptoms, eliminate the causation of symptoms either on a temporary or permanent basis, or to prevent or slow the appearance of symptoms of the named disorder or condition.

### Therapeutic Methods

In one aspect, methods for improving organ or tissue transplant survival, or delaying or preventing the onset of transplant rejection in a subject are provided. In some embodiments, the methods comprise administering to the subject one or more doses of an antibody that specifically binds to interleukin 2 receptor gamma chain (IL-2Rγ) prior to an organ or tissue transplant in the subject. In some embodiments, the methods for improving organ or tissue transplant survival, or delaying or preventing the onset of transplant rejection, comprise administering an IL-2Rγ antibody or antigen-binding fragment thereof in combination with one or more maintenance immunosuppressive therapies, e.g., an antiproliferative agent or a T cell costimulatory pathway blocker. In some embodiments, the methods comprise administering to the subject a combination therapy comprising an IL-2Rγ antibody or antigen-binding fragment thereof and a CD28 signaling inhibitor. In some embodiments, the methods comprise administering to the subject a combination therapy comprising an IL-2Rγ antibody or antigen-binding fragment thereof and a fusion protein comprising an extracellular portion of cytotoxic T lymphocyte-associated protein 4 (CTLA4) and an Fc region of immunoglobulin IgG1 (also referred to herein as “CTLA4-Ig”). In some embodiments, the methods comprise administering to the subject a combination therapy comprising an IL-2Rγ antibody or antigen-binding fragment thereof and one or more additional therapeutic agents, e.g., an antiproliferative agent such as mycophenolate mofetil.

In some embodiments, the method of improving organ or tissue transplant survival or delaying or preventing the onset of transplant rejection comprises: administering to the subject at least one dose of an antibody that specifically binds to human interleukin 2 receptor gamma chain (IL-2Rγ), or an antigen-binding fragment thereof, prior to an organ or tissue transplant in the subject, wherein the IL-2Rγ antibody or antigen-binding fragment thereof comprises three HCDRs (HCDR1, HCDR2 and HCDR3) and three LCDRs (LCDR1, LCDR2 and LCDR3), wherein the HCDR1 comprises the amino acid sequence of SEQ ID NO:2, the HCDR2 comprises the amino acid sequence of SEQ ID NO:3, the HCDR3 comprises the amino acid sequence of SEQ ID NO:4, the LCDR1 comprises the amino acid sequence of SEQ ID NO:6, the LCDR2 comprises the amino acid sequence AAS, and the LCDR3 comprises the amino acid sequence of SEQ ID NO:8.

In some embodiments, the method of improving organ or tissue transplant survival or delaying or preventing the onset of transplant rejection comprises: administering to the subject at least one dose of an antibody that specifically binds to human IL-2Rγ, or an antigen-binding fragment thereof, prior to an organ or tissue transplant in the subject, wherein the IL-2Rγ antibody or antigen-binding fragment thereof comprises three HCDRs (HCDR1, HCDR2 and HCDR3) and three LCDRs (LCDR1, LCDR2 and LCDR3), wherein the HCDR1 comprises the amino acid sequence of SEQ ID NO:2, the HCDR2 comprises the amino acid sequence of SEQ ID NO: 3, the HCDR3 comprises the amino acid sequence of SEQ ID NO:4, the LCDR1 comprises the amino acid sequence of SEQ ID NO:6, the LCDR2 comprises the amino acid sequence AAS, and the LCDR3 comprises the amino acid sequence of SEQ ID NO:8; and administering to the subject one or more doses of a CD28 signaling inhibitor (e.g., a CTLA4-Ig fusion protein).

In some embodiments, the method of improving organ or tissue transplant survival or delaying or preventing the onset of transplant rejection comprises: administering to the subject at least one dose of an antibody that specifically binds to human IL-2Rγ, or an antigen-binding fragment thereof, prior to an organ or tissue transplant in the subject, wherein the IL-2Rγ antibody or antigen-binding fragment thereof comprises three HCDRs (HCDR1, HCDR2 and HCDR3) and three LCDRs (LCDR1, LCDR2 and LCDR3), wherein the HCDR1 comprises the amino acid sequence of SEQ ID NO:2, the HCDR2 comprises the amino acid sequence of SEQ ID NO: 3, the HCDR3 comprises the amino acid sequence of SEQ ID NO:4, the LCDR1 comprises the amino acid sequence of SEQ ID NO:6, the LCDR2 comprises the amino acid sequence AAS, and the LCDR3 comprises the amino acid sequence of SEQ ID NO:8; and administering to the subject one or more doses of an antiproliferative agent (e.g., mycophenolate mofetil).

In some embodiments, the organ or tissue transplant is a bone transplant, corneal transplant, heart transplant, heart valve transplant, kidney transplant, ligament transplant, liver transplant, lung transplant, pancreas transplant, skin transplant, tendon transplant, trachea transplant, or vascular tissue transplant.

In some embodiments, multiple doses of the IL-2Rγ antibody or antigen-binding fragment thereof are administered to the subject prior to the transplant. In some embodiments, the IL-2Rγ antibody or antigen-binding fragment thereof is administered to the subject prior to the transplant (e.g., 1, 2, 3, 4 or more doses prior to the transplant) and after the transplant. In some embodiments, the IL-2Rγ antibody or antigen-binding fragment thereof is administered to the subject starting at least one day before the transplant (e.g., at least 2 days, at least 3 days, at least 4 days, at least 5 days, at least 6 days, or at least 7 days before the transplant). In some embodiments, the IL-2Rγ antibody or antigen-binding fragment thereof is administered to the subject as a prophylactic treatment for an extended period of time prior to the transplant (e.g., for at least 2 weeks, at least 3 weeks, at least 4 weeks, at least 5 weeks, at least 6 weeks, or longer). In some embodiments, the IL-2Rγ antibody or antigen-binding fragment thereof is administered to the subject no earlier than 10 days, 9 days, 8 days, 7 days, 6 days, 5 days, 4 days, 3 days, 2 days, or 1 day before the transplant.

In some embodiments, the IL-2Rγ antibody or antigen-binding fragment thereof is administered to the subject for at least 1, 2, 3, 4, 5, 6, 7, 8, 9, 10, 11, 12 months or more after the transplant. In some embodiments, the IL-2Rγ antibody or antigen-binding fragment thereof is administered for at least 1 year, at least 2 years, or more after the transplant. In some embodiments, the IL-2Rγ antibody or antigen-binding fragment thereof is administered until the onset of transplant rejection, or until complete transplant rejection.

Combination Therapy with a T Cell Costimulatory Pathway Blocker

In some embodiments, the IL-2Rγ antibody or antigen-binding fragment thereof is administered to the subject in combination with one or more T cell costimulatory pathway blockers. In some embodiments, the combination therapy comprises administration of a CD28 signaling inhibitor and/or a CD40L signaling inhibitor.

In some embodiments, the CD28 signaling inhibitor (e.g., CTLA4-Ig fusion protein) is administered to the subject as a maintenance therapy starting after the subject receives the transplant. In some embodiments, the CD28 signaling inhibitor is administered to the subject starting on the day of the organ or tissue transplant. In some embodiments, the CD28 signaling inhibitor is administered to the subject starting at least one day before the transplant (e.g., at least 2 days, at least 3 days, at least 4 days, at least 5 days, at least 6 days, or at least 7 days before the transplant). In some embodiments, the CD28 signaling inhibitor is administered to the subject for at least 1, 2, 3, 4, 5, 6, 7, 8, 9, 10, 11, 12 months or more after the transplant, or until the onset of transplant rejection, or until complete transplant rejection.

In some embodiments, the methods of the disclosure comprise administering an IL-2Rγ antibody or antigen-binding fragment thereof to a subject who is taking or has been prescribed a CD28 signaling inhibitor. In some embodiments, the subject is taking or has been prescribed a CTLA4-Ig fusion protein, e.g., abatacept or belatacept. In some embodiments, the subject is taking or has been prescribed abatacept in an approved dosing regimen (e.g., at a 10 mg/kg dose or a 15 mg/kg dose as a 60-minute infusion on the day before transplantation, followed by one or more doses after transplant, such as a 10 mg/kg or 12 mg/kg dose on Day 5, 14, and 28 after transplant). In some embodiments, the subject is taking or has been prescribed belatacept in an approved dosing regimen (e.g., at a 10 mg/kg dose on the day of transplantation prior to implantation, followed by one or more doses after transplant, such as a 10 mg/kg dose on Day 5 and at the end of Week 2, Week 4, Week 8, and Week 12, optionally followed by one or more maintenance doses, such as a 5 mg/kg dose at the end of Week 16 and every 4 weeks afterwards).

In some embodiments, the method comprises administering one or more additional therapeutic agents. For example, in some embodiments, the method comprises administering an IL-2Rγ antibody or antigen-binding fragment thereof and a CD40L inhibitor, or administering an IL-2Rγ antibody or antigen-binding fragment thereof, CD28 signaling inhibitor, and a CD40L inhibitor. In some embodiments, the additional therapeutic agent(s) (e.g., a CD40L inhibitor) is administered to the subject starting on the day of the organ or tissue transplant. In some embodiments, the additional therapeutic agent(s) is administered to the subject starting at least one day before the transplant (e.g., at least 2 days, at least 3 days, at least 4 days, at least 5 days, at least 6 days, or at least 7 days before the transplant). In some embodiments, the additional therapeutic agent(s) is administered to the subject for at least 1, 2, 3, 4, 5, 6, 7, 8, 9, 10, 11, 12 months or more after the transplant, or until the onset of transplant rejection, or until complete transplant rejection.

In some embodiments, wherein the method comprises administering to the subject an IL-2Rγ antibody or antigen-binding fragment thereof, a CD28 signaling inhibitor, and a CD40L inhibitor, one or more of the IL-2Rγ antibody or antigen-binding fragment thereof, CD28 signaling inhibitor, the CD40L inhibitor is administered to the subject until the onset of transplant rejection, or until complete transplant rejection. In some embodiments, each of the IL-2Rγ antibody or antigen-binding fragment thereof, CD28 signaling inhibitor, the CD40L inhibitor is administered to the subject until the onset of transplant rejection, or until complete transplant rejection.

Combination Therapy with an Antiproliferative Agent

In some embodiments, the IL-2Rγ antibody or antigen-binding fragment thereof is administered to the subject in combination with one or more antiproliferative agents, such as an agent that inhibits de novo synthesis of purines. In some embodiments, the combination therapy comprises administration of the antiproliferative agent mycophenolate (e.g., mycophenolate mofetil (MMF) or mycophenolate sodium), azathioprine (AZA), or leflunomide.

In some embodiments, the antiproliferative agent (e.g., MMF) is administered to the subject as a maintenance therapy starting after the subject receives the transplant. In some embodiments, the antiproliferative agent is administered to the subject starting on the day of the organ or tissue transplant. In some embodiments, the antiproliferative agent is administered to the subject starting at least one day before the transplant (e.g., at least 2 days, at least 3 days, at least 4 days, at least 5 days, at least 6 days, or at least 7 days before the transplant). In some embodiments, the antiproliferative agent is administered to the subject for at least 1, 2, 3, 4, 5, 6, 7, 8, 9, 10, 11, 12 months or more after the transplant, or until the onset of transplant rejection, or until complete transplant rejection.

In some embodiments, the methods of the disclosure comprise administering an IL-2Rγ antibody or antigen-binding fragment thereof to a subject who is taking or has been prescribed an antiproliferative agent. In some embodiments, the subject is taking or has been prescribed an antiproliferative agent that inhibits de novo synthesis of purines, e.g., MMF or AZA. In some embodiments, the subject is taking or has been prescribed an antiproliferative agent in an approved dosing regimen.

### IL-2 Receptor Gamma Chain Antibodies and Antigen-Binding Fragments Thereof

In one aspect, the methods of the disclosure comprise administering one or more doses of an IL-2Rγ antibody or antigen-binding fragment thereof to a subject. In some embodiments, the antibody or antigen-binding fragment thereof specifically binds human IL-2Rγ. According to certain embodiments of the present disclosure, an IL-2Rγ antibody or antigen-binding fragment thereof comprises a heavy chain variable region (HCVR), light chain variable region (LCVR), and/or complementarity determining regions (CDRs) comprising any of the amino acid sequences of the IL-2Rγ antibodies as set forth in WO 2020/160242, incorporated by reference herein. In some embodiments, the IL-2Rγ antibody is H4H12857P, H4H12858P, H4H12859P, H4H12863P, H4H12874P, H4H12871P, H4H12884P, H4H12886P, H4H12889P (REGN7257), H4H12890P, H4H12899P, H4H12900P, H4H12908P, H4H12913P2, H4H12922P2, H4H12924P2, H4H12926P2, H4H12927P2, H4H12934P2, H4H13538P, H4H13541P, H4H13544P2, or H4H13545P2, as set forth in WO 2020/160242.

In some embodiments, an IL-2Rγ antibody or antigen-binding fragment thereof comprises the heavy chain complementarity determining regions (HCDRs) of a heavy chain variable region (HCVR) comprising the amino acid sequence of SEQ ID NO: 1 and the light chain complementarity determining regions (LCDRs) of a light chain variable region (LCVR) comprising the amino acid sequence of SEQ ID NO:5. In some embodiments, the IL-2Rγ antibody or antigen-binding fragment thereof comprises three HCDRs (HCDR1, HCDR2 and HCDR3) and three LCDRs (LCDR1, LCDR2 and LCDR3), wherein the HCDR1 comprises or consists of the amino acid sequence of SEQ ID NO:2, the HCDR2 comprises or consists of the amino acid sequence of SEQ ID NO:3, the HCDR3 comprises or consists of the amino acid sequence of SEQ ID NO:4, the LCDR1 comprises or consists of the amino acid sequence of SEQ ID NO:6, the LCDR2 comprises or consists of the amino acid sequence AAS, and the LCDR3 comprises or consists of the amino acid sequence of SEQ ID NO:8.

In some embodiments, the IL-2Rγ antibody or antigen-binding fragment thereof comprises an HCDR1 comprising or consisting of the amino acid sequence of SEQ ID NO:2, an HCDR2 comprising or consisting the amino acid sequence of SEQ ID NO:3, and an HCDR3 comprising or consisting the amino acid sequence of SEQ ID NO:4, and further comprises an HCVR having at least 85% sequence identity (e.g., at least 90%, 91%, 92%, 93%, 94%, 95%, 96%, 97%, 98%, or 99% sequence identity) to the amino acid sequence of SEQ ID NO:1. In some embodiments, the IL-2Rγ antibody or antigen-binding fragment thereof comprises an LCDR1 comprising or consisting of the amino acid sequence of SEQ ID NO:6, an LCDR2 comprising or consisting the amino acid sequence AAS, and an LCDR3 comprising or consisting the amino acid sequence of SEQ ID NO:8, and further comprises an LCVR having at least 85% sequence identity (e.g., at least 90%, 91%, 92%, 93%, 94%, 95%, 96%, 97%, 98%, or 99% sequence identity) to the amino acid sequence of SEQ ID NO:5.

In some embodiments, the IL-2Rγ antibody or antigen-binding fragment thereof comprises an HCVR comprising, consisting essentially of, or consisting of SEQ ID NO:1, and an LCVR comprising, consisting essentially of, or consisting of SEQ ID NO:5.

In some embodiments, the IL-2Rγ antibody or antigen-binding fragment thereof comprises a heavy chain having at least 85% sequence identity (e.g., at least 90%, 91%, 92%, 93%, 94%, 95%, 96%, 97%, 98%, or 99% sequence identity) to the amino acid sequence of SEQ ID NO:9. In some embodiments, the IL-2Rγ antibody or antigen-binding fragment thereof comprises a heavy chain comprising, consisting essentially of, or consisting of the amino acid sequence of SEQ ID NO:9. In some embodiments, the IL-2Rγ antibody or antigen-binding fragment thereof comprises a light chain having at least 85% sequence identity (e.g., at least 90%, 91%, 92%, 93%, 94%, 95%, 96%, 97%, 98%, or 99% sequence identity) to the amino acid sequence of SEQ ID NO: 10. In some embodiments, the IL-2Rγ antibody or antigen-binding fragment thereof comprises a light chain comprising, consisting essentially of, or consisting of the amino acid sequence of SEQ ID NO: 10.

In some embodiments, the IL-2Rγ antibody is REGN7257 or a bioequivalent thereof.

### CD28 Signaling Inhibitors

In some embodiments, the methods comprise administering to a subject one or more doses of a CD28 signaling inhibitor. In some embodiments, a CD28 signaling inhibitor is a molecule (e.g., antigen-binding protein or small molecule) that directly binds to CD28 and inhibits CD28 signaling. In some embodiments, the CD28 signaling inhibitor is an antibody or antigen-binding fragment that specifically binds CD28. Exemplary CD28 antibodies that can be used in the methods of the disclosure are described in U.S. Pat. Nos. 8,785,604; 9,085,629; 10,919,965.

In some embodiments, a CD28 signaling inhibitor is a molecule (e.g., antigen-binding protein or small molecule) that indirectly inhibits CD28 signaling. For example, in some embodiments, the CD28 signaling inhibitor binds to one or more ligands of CD28 (e.g., CD80 and/or CD86) and prevents the engagement of the ligand(s) with CD28.

In some embodiments, the CD28 signaling inhibitor is CTLA4, or a mutated or truncated form of CTLA4, or a fusion protein comprising a portion of CTLA4 (e.g., a fusion protein comprising the extracellular domain of CTLA4) that can bind to CD80 and CD86 and prevent the engagement of CD80 and CD86 with the T cell costimulatory CD28. In some embodiments, the CD28 signaling inhibitor is a soluble form of CTLA4, e.g., a soluble mutated CTLA4. In some embodiments, the soluble CTLA4 or soluble mutated CTLA4 does not have a transmembrane domain. In some embodiments, a soluble mutated CTLA4 comprises one or more mutations (e.g., substitutions, additions, or deletions) that results in improved binding avidity for CD80 and/or CD86, e.g., relative to wild-type CTLA4. Exemplary soluble forms of CTLA4 that can be used in the methods of the disclosure are described in U.S. Pat. No. 7,482,327 and in patent publication WO2020/088645.

In some embodiments, the CD28 signaling inhibitor is a CTLA4-Ig fusion protein. In some embodiments, a CTLA4-Ig fusion protein comprises all or a portion of the extracellular domain of CTLA4 linked to a human immunoglobulin constant region or portion thereof (e.g., from IgG1). In some embodiments, the fusion protein comprises the entire extracellular domain of CTLA4. In some embodiments, the fusion protein comprises a portion of the extracellular domain of CTLA4, e.g., a portion of the CTLA4 extracellular domain that binds to CD80 and/or CD86. In some embodiments, the fusion protein comprises a wild-type (i.e., naturally occurring) CTLA4 extracellular domain or portion thereof. In some embodiments, the fusion protein comprises a CTLA4 extracellular domain or portion thereof into which one or more mutations (e.g., substitutions, additions, or deletions) have been introduced. In some embodiments, the mutated CTLA4 extracellular domain or portion thereof exhibits improved binding avidity for CD80 and/or CD86, e.g., relative to wild-type CTLA4. In some embodiments, the fusion protein comprises the hinge, CH2 and CH3 regions of human IgG1.

Exemplary CTLA4-Ig fusion proteins that can be used in the methods of the disclosure are described in U.S. Pat. Nos. 5,851,795; 6,444,792; 7,094,874; and 11,261,233; in patent publications US2018/0208639, US2022/0348634, and WO2013/041029; and in Douthwaite et al., J Immunol 2017, 198:528-537. In some embodiments, the CTLA4-Ig fusion protein is abatacept, belatacept, or MEDI5275 (AstraZeneca).

In some embodiments, the CD28 signaling inhibitor is abatacept, belatacept, MEDI5275, lulizumab pegol, FR104 (OSE Immunotherapeutics), or a bioequivalent thereof.

### CD40L Inhibitors

In some embodiments, the methods comprise administering to a subject one or more doses of an inhibitor of CD40 ligand (“CD40L”; also known in the art as CD154). In some embodiments, the CD40L inhibitor is administered prior to transplant (e.g., at least one day prior to transplant). In some embodiments, the CD40L inhibitor is first administered on the day of transplant or after transplant. In some embodiments, the CD40L inhibitor is first administered prior to transplant (e.g., at least one day prior to transplant) and administration is continued after transplant (e.g., until transplant rejection).

In some embodiments, the CD40L inhibitor is an antigen-binding protein that specifically binds to CD40L, e.g., an antibody that specifically binds to CD40L or an antigen-binding fragment thereof, a modified antibody or antigen-binding fragment that specifically binds CD40L (e.g., having an Fc modification), or a polypeptide or fusion protein that specifically binds to CD40L. Exemplary CD40L antigen-binding proteins that can be used in the methods of the disclosure are described in U.S. Pat. Nos. 7,445,781; 8,293,237; 8,647,625; 8,895,010; 8,961,976; 10,000,553; 11,384,152; and 11,596,689; and in patent publications US2013/0095109, US2017/0051059, WO2016/126702, WO2017/011544, and WO2018/217918.

In some embodiments, the CD40L inhibitor is a small molecule inhibitor, e.g., a small molecule inhibitor of CD40-CD40L interaction. Exemplary CD40L antigen-binding proteins that can be used in the methods of the disclosure are described in Silvian et al., ACS Chem Biol 2011, 6:636-647; Chen et al., J Med Chem 2017, 60:8906-8922; and Karnell et al, Advanced Drug Delivery Reviews 2019, 141:92-103.

In some embodiments, the CD40L inhibitor is dapirolizumab pegol, dazodalibep, frexalimab, letolizumab, ruplizumab, tegoprubart, or MR-1 (BioXCell #BE0017-1).

In some embodiments, the methods of the present disclosure comprise administering to the subject an IL-2Rγ antibody, a CD28 signaling inhibitor, and a CD40L inhibitor. In some embodiments, the IL-2Rγ antibody is H4H12857P, H4H12858P, H4H12859P, H4H12863P, H4H12874P, H4H12871P, H4H12884P, H4H12886P, H4H12889P (REGN7257), H4H12890P, H4H12899P, H4H12900P, H4H12908P, H4H12913P2, H4H12922P2, H4H12924P2, H4H12926P2, H4H12927P2, H4H12934P2, H4H13538P, H4H13541P, H4H13544P2, or H4H13545P2, as set forth in WO 2020/160242. In some embodiments, the IL-2Rγ antibody is REGN7257.

In some embodiments, the methods of the present disclosure comprise administering to the subject an IL-2Rγ antibody, a CTLA4-Ig fusion protein, and a CD40L inhibitor. In some embodiments, the IL-2Rγ antibody is H4H12857P, H4H12858P, H4H12859P, H4H12863P, H4H12874P, H4H12871P, H4H12884P, H4H12886P, H4H12889P (REGN7257), H4H12890P, H4H12899P, H4H12900P, H4H12908P, H4H12913P2, H4H12922P2, H4H12924P2, H4H12926P2, H4H12927P2, H4H12934P2, H4H13538P, H4H13541P, H4H13544P2, or H4H13545P2, as set forth in WO 2020/160242; the CTLA4-Ig fusion protein is abatacept or belatacept; and the CD40L inhibitor is dapirolizumab pegol, dazodalibep, frexalimab, letolizumab, ruplizumab, tegoprubart, or MR-1. In some embodiments, the IL-2Rγ antibody is H4H12889P (REGN7257); the CTLA4-Ig fusion protein is abatacept or belatacept; and the CD40L inhibitor is dapirolizumab pegol, dazodalibep, frexalimab, letolizumab, ruplizumab, tegoprubart, or MR-1. In some embodiments, the IL-2Rγ antibody is H4H12889P (REGN7257); the CTLA4-Ig fusion protein is abatacept or belatacept; and the CD40L inhibitor is MR-1.

### Additional Therapeutic Agents

In some embodiments, the methods comprise administering an IL-2Rγ antibody or antigen-binding fragment thereof, or a combination therapy comprising an IL-2Rγ antibody or antigen-binding fragment thereof and a CD28 signaling inhibitor, in further combination with one or more additional therapeutic agents. In some embodiments, the additional therapeutic agent(s) is an immunomodulatory agent or anti-inflammatory agent. In some embodiments, the additional therapeutic agent(s) is immunosuppressive therapy.

Exemplary additional therapeutic agents include, e.g., a steroid (e.g., corticosteroids such as topical, systemic, oral, or inhaled corticosteroids, including but not limited to betamethasone, clobetasol, dexamethasone, fluocinolone, fluocinonide, halobetasol, hydrocortisone, methylprednisolone, prednisone, prednisolone, or triamcinolone); a non-steroidal topical medication such as but not limited to a PDE4 inhibitor or a calcineurin inhibitor; a non-steroidal anti-inflammatory drug (NSAID) such as but not limited to celecoxib, diclofenac, etodolac, fenprofen, flurbiprofen, ibuprofen, ketoprofen, meclofamate, meloxicam, nabumetone, naproxen, oxaprozin, piroxicam, rofecoxib, salicylates, sulfasalazinem, sulindac, or tolmetin; an anti-inflammatory antibody or biologic (e.g., a TNFα antibody or biologic such as but not limited to adalimumab, certolizumab, etanercept, golimumab, or infliximab; an IL1 antibody or biologic such as but not limited to LY2189102, anakinra, canakinumab, gerokizumab, or rilonacept; an IL6/IL-6R antibody or biologic such as but not limited to sarilumab, siltuximab, or tocilizumab; an IL17A/IL-17R antibody or biologic such as but not limited to bimekizumab, brodalumab, ixekizumab, or secukinumab; a CD40 antibody or biologic such as but not limited to iscalimab; or an IL12/IL-23 antibody or biologic such as but not limited to AMG139, BI655066, brazikumab, briankizumab, guselkumab, mirikizumab, risankizumab, tildrakizumab, or ustekinumab); a JAK inhibitor such as but not limited to abrocitinib, baricitinib, fedratinib, filgotinib, ruxolitinib, tofacitinib, or upadacitinib; an immunosuppressive agent (e.g., a systemic immunosuppressant such as but not limited to methotrexate, cyclophosphamide, mizoribine, chlorambucil, cyclosporine, mycophenolate mofetil, or azathioprine); a disease-modifying antirheumatic drug (DMARD) such as but not limited to apremilast, azathioprine, baricitinib, cyclophosphamide, cyclosporine, hydroxychloroquine, leflunomide, methotrexate, mycophenolate mofetil, sulfasalazine, or tofacitinib; radiation therapy; chemotherapy; intravenous immunoglobulin therapy.

In some embodiments, the additional therapeutic agent(s) is an immunosuppressive agent. In some embodiments, the immunosuppressive agent is an antiproliferative agent, e.g., an agent that inhibits de novo synthesis of purines or an mTOR inhibitor. In some embodiments, the additional therapeutic agent(s) is mycophenolate (e.g., mycophenolate mofetil [also referred to herein as “MMF”] or mycophenolate sodium), AZA, or leflunomide. In some embodiments, the additional therapeutic agent is MMF.

The additional therapeutically active component(s) may be administered prior to, concurrent with, or shortly after the administration of an IL-2Rγ antibody or combination therapy comprising an IL-2Rγ antibody as disclosed herein.

In some embodiments, the additional therapeutic agent(s) are administered to the subject as a maintenance therapy starting after the subject receives the transplant. In some embodiments, the additional therapeutic agent(s) are administered to the subject starting on the day of the organ or tissue transplant. In some embodiments, the additional therapeutic agent(s) are administered to the subject starting at least one day before the transplant (e.g., at least 2 days, at least 3 days, at least 4 days, at least 5 days, at least 6 days, or at least 7 days before the transplant). In some embodiments, the additional therapeutic agent(s) are administered to the subject for at least 1, 2, 3, 4, 5, 6, 7, 8, 9, 10, 11, 12 months or more after the transplant, or until the onset of transplant rejection, or until complete transplant rejection.

### Preparation of Human Antibodies

Methods for generating human antibodies in transgenic mice are known in the art. Any such known methods can be used in the context of the present disclosure to make human antibodies that specifically bind to IL-2Rγ.

Using VELOCIMMUNE™ technology (see, for example, U.S. Pat. No. 6,596,541, Regeneron Pharmaceuticals) or any other known method for generating monoclonal antibodies, high affinity chimeric antibodies to IL-4R are initially isolated having a human variable region and a mouse constant region. The VELOCIMMUNE® technology involves generation of a transgenic mouse having a genome comprising human heavy and light chain variable regions operably linked to endogenous mouse constant region loci such that the mouse produces an antibody comprising a human variable region and a mouse constant region in response to antigenic stimulation. The DNA encoding the variable regions of the heavy and light chains of the antibody are isolated and operably linked to DNA encoding the human heavy and light chain constant regions. The DNA is then expressed in a cell capable of expressing the fully human antibody.

Generally, a VELOCIMMUNE® mouse is challenged with the antigen of interest, and lymphatic cells (such as B-cells) are recovered from the mice that express antibodies. The lymphatic cells may be fused with a myeloma cell line to prepare immortal hybridoma cell lines, and such hybridoma cell lines are screened and selected to identify hybridoma cell lines that produce antibodies specific to the antigen of interest. DNA encoding the variable regions of the heavy chain and light chain may be isolated and linked to desirable isotypic constant regions of the heavy chain and light chain. Such an antibody protein may be produced in a cell, such as a CHO cell. Alternatively, DNA encoding the antigen-specific chimeric antibodies or the variable domains of the light and heavy chains may be isolated directly from antigen-specific lymphocytes.

Initially, high affinity chimeric antibodies are isolated having a human variable region and a mouse constant region. The antibodies are characterized and selected for desirable characteristics, including affinity, selectivity, epitope, etc., using standard procedures known to those skilled in the art. The mouse constant regions are replaced with a desired human constant region to generate the fully human antibody of the disclosure, for example wild-type or modified IgG1 or IgG4. While the constant region selected may vary according to specific use, high affinity antigen-binding and target specificity characteristics reside in the variable region.

In general, the antibodies that can be used in the methods of the present disclosure possess high affinities, as described above, when measured by binding to antigen either immobilized on solid phase or in solution phase. The mouse constant regions are replaced with desired human constant regions to generate the fully human antibodies of the disclosure. While the constant region selected may vary according to specific use, high affinity antigen-binding and target specificity characteristics reside in the variable region.

Methods and techniques for identifying CDRs within HCVR and LCVR amino acid sequences are well known in the art and can be used to identify CDRs within the specified HCVR and/or LCVR amino acid sequences disclosed herein. Exemplary conventions that can be used to identify the boundaries of CDRs include, e.g., the Kabat definition, the Chothia definition, and the AbM definition. In general terms, the Kabat definition is based on sequence variability, the Chothia definition is based on the location of the structural loop regions, and the AbM definition is a compromise between the Kabat and Chothia approaches. See, e.g., Kabat, “Sequences of Proteins of Immunological Interest,” National Institutes of Health, Bethesda, Md. (1991); Al-Lazikani et al., J. Mol. Biol. 273:927-948 (1997); and Martin et al., Proc. Natl. Acad. Sci. USA 86:9268-9272 (1989). Public databases are also available for identifying CDR sequences within an antibody.

### Bioequivalents

The present disclosure encompasses antigen-binding molecules (e.g., antibodies and fusion proteins) having amino acid sequences that vary from those of the described antibodies but that retain the ability to bind a target antigen (e.g., IL-2Rγ). Such variant molecules comprise one or more additions, deletions, or substitutions of amino acids when compared to the parent sequence, but exhibit biological activity that is essentially equivalent to that of the described antibodies. Likewise, the nucleic acid sequences encoding the antigen-binding molecules (e.g., antibodies and fusion proteins) encompass sequences that comprise one or more additions, deletions, or substitutions of nucleotides when compared to the disclosed sequence, but that encode an antigen-binding molecule that is essentially bioequivalent to the antigen-binding molecules disclosed herein.

The present disclosure includes antigen-binding molecules that are bioequivalent to any of the exemplary antigen-binding molecules set forth herein. Two antigen-binding molecules are considered bioequivalent if, for example, they are pharmaceutical equivalents or pharmaceutical alternatives whose rate and extent of absorption do not show a significant difference when administered at the same molar dose under similar experimental conditions, either single does or multiple dose. Some antigen-binding molecules will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied.

In one embodiment, two antigen-binding molecules are bioequivalent if there are no clinically meaningful differences in their safety, purity, and potency.

In one embodiment, two antigen-binding molecules are bioequivalent if a patient can be switched one or more times between the first antigen-binding molecule (e.g., reference product) and the second antigen-binding molecule (e.g., biological product) without an expected increase in the risk of adverse effects, including a clinically significant change in immunogenicity, or diminished effectiveness, as compared to continued therapy without such switching.

In one embodiment, two antigen-binding molecules are bioequivalent if they both act by a common mechanism or mechanisms of action for the condition or conditions of use, to the extent that such mechanisms are known.

Bioequivalence may be demonstrated by in vivo and in vitro methods. Non-limiting examples of bioequivalence measures include, e.g., (a) an in vivo test in humans or other mammals, in which the concentration of the antibody or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antibody (or its target) is measured as a function of time; and (d) in a well-controlled clinical trial that establishes safety, efficacy, or bioavailability or bioequivalence of an antibody.

Bioequivalent variants of the antigen-binding molecules set forth herein may be constructed by, for example, making various substitutions of residues or sequences or deleting terminal or internal residues or sequences not needed for biological activity. For example, cysteine residues not essential for biological activity can be deleted or replaced with other amino acids to prevent formation of unnecessary or incorrect intramolecular disulfide bridges upon renaturation. In some embodiments, bioequivalent antibodies may include the exemplary antibodies set forth herein comprising amino acid changes which modify the glycosylation characteristics of the antibodies, e.g., mutations which eliminate or remove glycosylation.

### Pharmaceutical Compositions, Combinations, and Kits

In another aspect, the present disclosure provides pharmaceutical compositions comprising an IL-2Rγ antibody, or an IL-2Rγ antibody in combination with one or more additional therapeutic agents (e.g., a CD28 signaling inhibitor such as a CTLA4-Ig fusion protein, and/or a CD40L inhibitor, and/or an antiproliferative agent such as MMF). The pharmaceutical compositions are formulated with one or more pharmaceutically acceptable vehicle, carriers, and/or excipients. Various pharmaceutically acceptable carriers and excipients are well-known in the art. See, e.g., Remington's Pharmaceutical Sciences, Mack Publishing Company, Easton, PA. In some embodiments, the carrier is suitable for intravenous, intramuscular, oral, intraperitoneal, intrathecal, transdermal, topical, or subcutaneous administration.

In some embodiments, the pharmaceutical composition comprises an injectable preparation, such as a dosage form for intravenous, subcutaneous, intracutaneous and intramuscular injections, drip infusions, etc. These injectable preparations may be prepared by known methods. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared can be filled in an appropriate ampoule.

Various delivery systems are known and can be used to administer the pharmaceutical composition, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Methods of administration include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. In some embodiments, a pharmaceutical composition as disclosed herein is administered intravenously. In some embodiments, a pharmaceutical composition as disclosed herein is administered subcutaneously.

In some embodiments, a pharmaceutical composition of the present disclosure is contained within a container. Thus, in another aspect, containers comprising a pharmaceutical composition as disclosed herein are provided. For example, in some embodiments, a pharmaceutical composition is contained within a container selected from the group consisting of a glass vial, a syringe, a pen delivery device, and an autoinjector.

In some embodiments, a pharmaceutical composition of the present disclosure is delivered, e.g., subcutaneously or intravenously, with a standard needle and syringe. In some embodiments, the syringe is a pre-filled syringe. In some embodiments, a pen delivery device or autoinjector is used to deliver a pharmaceutical composition of the present disclosure (e.g., for subcutaneous delivery). A pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Examples of suitable pen and autoinjector delivery devices include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, IN), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™, OPTIPEN STARLET™, and OPTICLIK™ (Sanofi-Aventis, Frankfurt, Germany). Examples of disposable pen delivery devices having applications in subcutaneous delivery of a pharmaceutical composition of the present disclosure include, but are not limited to the SOLOSTAR™ pen (Sanofi-Aventis), the FLEXPEN™ (Novo Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L. P.), and the HUMIRA™ Pen (Abbott Labs, Abbott Park IL).

In some embodiments, the pharmaceutical composition is delivered using a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Florida. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

In some embodiments, pharmaceutical compositions for use as described herein are prepared into dosage forms in a unit dose suited to fit a dose of the active ingredients. Such dosage forms in a unit dose include, for example, tablets, pills, capsules, injections (ampoules), suppositories, etc.

In another aspect, the present disclosure provides combinations comprising an IL-2Rγ antibody and a CD28 signaling inhibitor for use in improving organ or tissue transplant survival or for use in delaying the onset of transplant rejection. In some embodiments, the combination comprises (i) an IL-2Rγ antibody that is H4H12857P, H4H12858P, H4H12859P, H4H12863P, H4H12874P, H4H12871P, H4H12884P, H4H12886P, H4H12889P (REGN7257), H4H12890P, H4H12899P, H4H12900P, H4H12908P, H4H12913P2, H4H12922P2, H4H12924P2, H4H12926P2, H4H12927P2, H4H12934P2, H4H13538P, H4H13541P, H4H13544P2, or H4H13545P2, as set forth in WO 2020/160242; and (ii) a CTLA4-Ig fusion protein that is abatacept or belatacept. In some embodiments, the combination comprises (i) an IL-2Rγ antibody that is REGN7257; and (ii) a CTLA4-Ig fusion protein that is abatacept or belatacept.

In some embodiments, the combination further comprises a CD40L inhibitor. Thus, in some embodiments, the combination comprises (i) an IL-2Rγ antibody that is H4H12857P, H4H12858P, H4H12859P, H4H12863P, H4H12874P, H4H12871P, H4H12884P, H4H12886P, H4H12889P (REGN7257), H4H12890P, H4H12899P, H4H12900P, H4H12908P, H4H12913P2, H4H12922P2, H4H12924P2, H4H12926P2, H4H12927P2, H4H12934P2, H4H13538P, H4H13541P, H4H13544P2, or H4H13545P2, as set forth in WO 2020/160242; (ii) a CTLA4-Ig fusion protein that is abatacept or belatacept; and (iii) a CD40L inhibitor that is dapirolizumab pegol, dazodalibep, frexalimab, letolizumab, ruplizumab, tegoprubart, or MR-1. In some embodiments, the combination comprises (i) an IL-2Rγ antibody that is REGN7257; (ii) a CTLA4-Ig fusion protein that is abatacept or belatacept; and (iii) a CD40L inhibitor that is dapirolizumab pegol, dazodalibep, frexalimab, letolizumab, ruplizumab, tegoprubart, or MR-1. In some embodiments, the combination comprises (i) an IL-2Rγ antibody that is REGN7257; (ii) a CTLA4-Ig fusion protein that is abatacept or belatacept; and (iii) a CD40L inhibitor that is MR-1.

In another aspect, the present disclosure provides combinations comprising an IL-2Rγ antibody and an antiproliferative agent for use in improving organ or tissue transplant survival or for use in delaying the onset of transplant rejection. In some embodiments, the combination comprises (i) an IL-2Rγ antibody that is H4H12857P, H4H12858P, H4H12859P, H4H12863P, H4H12874P, H4H12871P, H4H12884P, H4H12886P, H4H12889P (REGN7257), H4H12890P, H4H12899P, H4H12900P, H4H12908P, H4H12913P2, H4H12922P2, H4H12924P2, H4H12926P2, H4H12927P2, H4H12934P2, H4H13538P, H4H13541P, H4H13544P2, or H4H13545P2, as set forth in WO 2020/160242; and (ii) an antiproliferative agent that is MMF, mycophenolate sodium, AZA, or leflunomide. In some embodiments, the combination comprises (i) an IL-2Rγ antibody that is REGN7257; and (ii) an antiproliferative agent that is MMF.

In another aspect, the present disclosure provides kits comprising an IL-2Rγ antibody or antigen-binding fragment thereof (e.g., REGN7257) or a pharmaceutical composition comprising an IL-2Rγ antibody (e.g., REGN7257) as disclosed herein, alone or in association with one or more additional components including, but not limited to, a further therapeutic agent, as discussed herein. In some embodiments, the kit is for use in improving organ or tissue transplant survival or for use in delaying the onset of transplant rejection.

In some embodiments, the kit includes an IL-2Rγ antibody or antigen-binding fragment thereof or a pharmaceutical composition comprising an IL-2Rγ antibody or antigen-binding fragment thereof as disclosed herein, in one container (e.g., in a sterile glass or plastic vial). In one embodiment, the kit further includes an additional therapeutic agent (e.g., a CD28 signaling inhibitor and/or CD40L inhibitor) in another container (e.g., in a sterile glass or plastic vial). In another embodiment, the kit comprises an IL-2Rγ antibody or antigen-binding fragment thereof or pharmaceutical composition as disclosed herein in combination with one or more further therapeutic agents in a single, common container.

In some embodiments, the kit comprises a device (e.g., an injection device) for administration. For example, the kit can include one or more hypodermic needles or other injection devices as discussed above containing the composition(s).

In some embodiments, the kit comprises a package insert including information concerning the composition(s) in the kit. Generally, such information aids patients and physicians in using the enclosed compositions effectively and safely. For example, the following information may be supplied in the insert: pharmacokinetics, pharmacodynamics, clinical studies, efficacy parameters, indications and usage, contraindications, warnings, precautions, adverse reactions, overdosage, proper dosage and administration, how supplied, proper storage conditions, references, manufacturer/distributor information, and patent information. In some embodiments, the kit comprises instructions for using the composition in the treatment of a disease, disorder, or condition as disclosed herein.

### Dosage and Administration Regimens

In some embodiments, an amount of a therapeutic agent as disclosed herein (e.g., an IL-2Rγ antibody or a CD28 signaling inhibitor) that is administered to a subject according to the methods disclosed herein is a therapeutically effective amount. As used herein, the phrase “therapeutically effective amount” means an amount that produces the desired effect for which it is administered.

In some embodiments, the therapeutic agent (e.g., an IL-2Rγ antibody or an additional therapeutic agent such as a CD28 signaling inhibitor) is administered to a subject as a weight-based dose. A “weight-based dose” (e.g., a dose in mg/kg) is a dose of the therapeutic agent that will change depending on the subject's weight.

In other embodiments, the therapeutic agent (e.g., an IL-2Rγ antibody or an additional therapeutic agent such as a CD28 signaling inhibitor) is administered as a fixed dose. A “fixed dose” (e.g., a dose in mg) means that one dose of the therapeutic agent is used for all subjects regardless of any specific subject-related factors, such as weight. In one particular embodiment, a fixed dose of a therapeutic agent is based on a predetermined weight or age.

Typically, a suitable dose of the therapeutic agent can be in the range of about 0.001 to about 200 milligram per kilogram body weight of the recipient, generally in the range of about 1 to 100 mg per kilogram body weight, e.g., 1-100 mg/kg, 1-50 mg/kg, 1-20 mg/kg, 5-100 mg/kg, 5-75 mg/kg, or 10-75 mg/kg. In some embodiments, the therapeutic agent (e.g., IL-2Rγ antibody, CD28 signaling inhibitor, CD40L inhibitor, or other therapeutic agent) is administered at a dose of about 0.05 mg to about 1000 mg, e.g., about 10-750 mg, about 20-600 mg, or about 25-500 mg. For example, the therapeutic agent can be administered at about 0.1 mg/kg, about 0.2 mg/kg, about 0.5 mg/kg, about 1 mg/kg, about 1.5 mg/kg, about 2 mg/kg, about 3 mg/kg, about 5 mg/kg, about 10 mg/kg, about 15 mg/kg, about 20 mg/kg, about 25 mg/kg, about 30 mg/kg, about 40 mg/kg, or about 50 mg/kg per single dose. Values and ranges intermediate to the recited values are also intended to be part of this disclosure.

In some embodiments, the therapeutic agent is administered as a fixed dose of between about 5 mg to about 2500 mg. In some embodiments, the therapeutic agent is administered as a fixed dose of about 5 mg, about 10 mg, about 15 mg, about 20 mg, about 25 mg, about 30 mg, about 50 mg, about 75 mg, about 100 mg, about 125 mg, about 150 mg, about 175 mg, 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1500 mg, about 2000 mg, or about 2500 mg. Values and ranges intermediate to the recited values are also intended to be part of this disclosure.

The dose administered to a patient according to the methods of the present disclosure may vary depending upon the age and the size of the patient, symptoms, conditions, route of administration, and the like. The dose is typically calculated according to body weight or body surface area. Depending on the severity of the condition, the frequency and the duration of the treatment can be adjusted. Effective dosages and schedules for administering the pharmaceutical compositions may be determined empirically; for example, patient progress can be monitored by periodic assessment, and the dose adjusted accordingly. Moreover, interspecies scaling of dosages can be performed using well-known methods in the art (e.g., Mordenti et al., 1991, Pharmaceut. Res. 8:1351).

In some embodiments, the therapeutic agent(s) is systemically administered to a subject. In some embodiments, the therapeutic agent(s) is administered to a subject subcutaneously. In some embodiments, the therapeutic agent(s) is administered to a subject intravenously.

In some embodiments, the therapeutic agent(s) is administered to a subject at a dosing frequency of about twice daily, once daily, six times a week, five times a week, four times a week, three times a week, twice a week, once a week, once every two weeks, once every three weeks, once every four weeks, once every five weeks, once every six weeks, once every eight weeks, once every twelve weeks, or less frequently so long as a therapeutic response is achieved.

In some embodiments, multiple doses of the therapeutic agent(s) are administered to a subject over a defined time course. In some embodiments, the methods of the present disclosure comprise sequentially administering to a subject multiple doses of a therapeutic agent (e.g., an IL-2Rγ antibody or a CD28 signaling inhibitor). As used herein, “sequentially administering” means that each dose of the therapeutic agent is administered to the subject at a different point in time, e.g., on different days separated by a predetermined interval (e.g., hours, days, weeks, or months). In some embodiments, the methods of the disclosure comprise sequentially administering to the patient a single initial dose of a therapeutic agent, followed by one or more secondary doses of the therapeutic agent, and optionally followed by one or more tertiary doses of the therapeutic agent. The terms “initial dose,” “secondary dose(s),” and “tertiary dose(s)” refer to the temporal sequence of administration of the therapeutic agent. Thus, the “initial dose” is the dose which is administered at the beginning of the treatment regimen (also referred to as the “loading dose”); the “secondary doses” are the doses which are administered after the initial dose; and the “tertiary doses” are the doses which are administered after the secondary doses.

In some embodiments, the initial, secondary, and tertiary doses all contain the same amount of the therapeutic agent, but differ from one another in terms of frequency of administration. In other embodiments, the amount of the therapeutic agent contained in the initial, secondary and/or tertiary doses varies from one another (e.g., adjusted up or down as appropriate) during the course of treatment. In certain embodiments, one or more (e.g., 1, 2, 3, 4, or 5) doses are administered at the beginning of the treatment regimen as “loading doses” followed by subsequent doses that are administered on a less frequent basis (e.g., “maintenance doses”). For example, the first amount of the therapeutic agent can be 1.5×, 2×, 2.5×, 3×, 3.5×, 4× or 5× or more than the second amount of the therapeutic agent.

In some embodiments, each secondary and/or tertiary dose is administered 1 to 30 (e.g., 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, 15, 15½, 16, 16½, 17, 17½, 18, 18½, 19, 19½, 20, 20½, 21, 21½, 22, 22½, 23, 23½, 24, 24½, 25, 25½, 26, 26½, 27, 27½, 28, 28½, 29, 29½, 30 or more) weeks after the immediately preceding dose. The phrase “the immediately preceding dose,” as used herein, means, in a sequence of multiple administrations, the dose of a therapeutic agent which is administered to a patient prior to the administration of the very next dose in the sequence with no intervening doses.

The methods of the disclosure may comprise administering to a patient any number of secondary and/or tertiary doses. For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In some embodiments involving multiple secondary doses, each secondary dose is administered at the same frequency as the other secondary doses. Similarly, in some embodiments involving multiple tertiary doses, each tertiary dose is administered at the same frequency as the other tertiary doses. Alternatively, the frequency at which the secondary and/or tertiary doses are administered to a patient can vary over the course of the treatment regimen. The frequency of administration may also be adjusted during the course of treatment by a physician depending on the needs of the individual patient following clinical examination.

A summary of the sequences and the corresponding SEQ ID NOs referenced herein is shown in Table 1, below.

TABLE 1
Sequence Listing

| SEQ ID | | |
|---|---|---|
| NO | Sequence | Description |
| 1 | EVQLVESGGGLVQPGGSLRLSCAASGFIFSSYEMHWVRQAPGKGLEWISYISSSG | REGN7257 heavy chain |
| | TTIYYADSVKGRFTISRDNAKNSLYLHMNSLRAEDTAVYYCTRARITGTFDVFDIW | variable region |
| | GQGTMVTVSS | |
| 2 | GFIFSSYE | REGN257 HCDR1 |
| 3 | ISSSGTTI | REGN257 HCDR2 |
| 4 | TRARITGTFDVFDI | REGN257 HCDR3 |
| 5 | DIQMTQSPSSLSASVGDRVTITCRASQSISSYLNWYQQKPGKAPKLLIFAASNLQS | REGN7257 light chain |
| | GVPSRFSGSRSGTDFTLTISSLQPEDFATYYCQQNYNIPYTFGQGTKLEIK | variable region |
| 6 | QSISSY | REGN257 LCDR1 |
| 7 | AAS | REGN257 LCDR2 |
| 8 | QQNYNIPYT | REGN257 LCDR3 |
| 9 | EVQLVESGGGLVQPGGSLRLSCAASGFIFSSYEMHWVRQAPGKGLEWISYISSSG | REGN7257 heavy chain |
| | TTIYYADSVKGRFTISRDNAKNSLYLHMNSLRAEDTAVYYCTRARITGTFDVFDIW | |
| | GQGTMVTVSSASTKGPSVFPLAPCSRSTSESTAALGCLVKDYFPEPVTVSWNSG | |
| | ALTSGVHTFPAVLQSSGLYSLSSVVTVPSSSLGTKTYTCNVDHKPSNTKVDKRVES | |
| | KYGPPCPPCPAPEFLGGPSVFLFPPKPKDTLMISRTPEVTCVVVDVSQEDPEVQF | |
| | NWYVDGVEVHNAKTKPREEQFNSTYRVVSVLTVLHQDWLNGKEYKCKVSNKGLP | |
| | SSIEKTISKAKGQPREPQVYTLPPSQEEMTKNQVSLTCLVKGFYPSDIAVEWESN | |
| | GQPENNYKTTPPVLDSDGSFFLYSRLTVDKSRWQEGNVFSCSVMHEALHNHYT | |
| | QKSLSLSLGK | |
| 10 | DIQMTQSPSSLSASVGDRVTITCRASQSISSYLNWYQQKPGKAPKLLIFAASNLQS | REGN7257 light chain |
| | GVPSRFSGSRSGTDFTLTISSLQPEDFATYYCQQNYNIPYTFGQGTKLEIKRTVAAP | |
| | SVFIFPPSDEQLKSGTASVVCLLNNFYPREAKVQWKVDNALQSGNSQESVTEQD | |
| | SKDSTYSLSSTLTLSKADYEKHKVYACEVTHQGLSSPVTKSFNRGEC | |

### EXAMPLES

The following examples are put forth so as to provide those of ordinary skill in the art with a complete disclosure and description of how to make and use the methods and compositions of the disclosure, and are not intended to limit the scope of what the inventors regard as their invention. Likewise, the disclosure is not limited to any particular preferred embodiments described herein. Indeed, modifications and variations of the embodiments may be apparent to those skilled in the art upon reading this specification and can be made without departing from its spirit and scope. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, room temperature is about 25° C., and pressure is at or near atmospheric.

### Example 1: IL-2Rγ Monotherapy Improves Graft Survival in Skin Transplant Model

An in vivo skin graft rejection model was used to assess the blocking activity of the IL-2Rγ antibody REGN7257. REGN7257 is a fully human IL-2Rγ antibody comprising the HCVR of SEQ ID NO: 1 and the LCVR of SEQ ID NO:5. REGN7257 is also known in the art as H4H12889P (see, WO 2020/160242). The HCVR, LCVR, and CDR sequences of REGN7257 are shown below:

HCVR:
(SEQ ID NO: 1)
EVQLVESGGGLVQPGGSLRLSCAASGFIFSSYEMHWVRQAPGKGL
EWISYISSSGTTIYYADSVKGRFTISRDNAKNSLYLHMNSLRAED
TAVYYCTRARITGTFDVEDIWGQGTMVTVSS
LCVR:
(SEQ ID NO: 5)
DIQMTQSPSSLSASVGDRVTITCRASQSISSYLNWYQQKPGKAPK
LLIFAASNLQSGVPSRESGSRSGTDFTLTISSLOPEDFATYYCQQ
NYNIPYTFGQGTKLEIK
HCDR1:
(SEQ ID NO: 2)
GFIFSSYE
HCDR2:
(SEQ ID NO: 3)
ISSSGTTI
HCDR3:
(SEQ ID NO: 4)
TRARITGTFDVFDI
LCDR1:
(SEQ ID NO: 6)
QSISSY
LCDR2:
AAS
LCDR3:
(SEQ ID NO: 8)
QQNYNIPYT

### Experimental Procedure:

BALB/cJ mice obtained from The Jackson Laboratory (Bar Harbor, ME) were used as skin graft donors, and MHC mismatched Velocigene® (VG) background mice (C57BL/6NTac (75%)/129S6SvEvTac (25%)) from the Regeneron Velocigene® breeding colony that were genetically modified to replace the endogenous IL2RG ectodomain with the corresponding human sequences were used as recipients. The skin graft was obtained from the tail of the donor mice. The skin was the peeled off using forceps and punched with a 10 mm diameter biopsy punch.

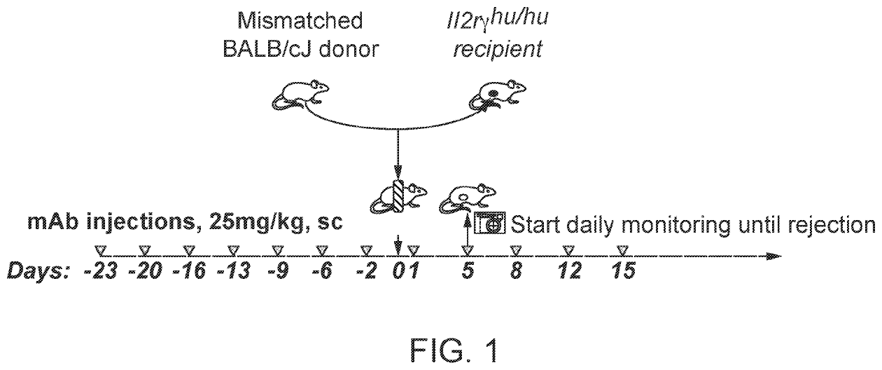

As detailed in Table 2 below, VG mice (humanized for IL2RG), used as graft recipients, were administered no treatment (negative control), an isotype control (REGN1945), or REGN7257 subcutaneously at doses 25 mg/kg at a frequency of 2 times per week starting 3 weeks prior to transplant, and continuing until rejection (FIG. 1). Recipients with the surgical site shaved were anesthetized by isoflurane via a nose cone and administered an analgesic (buprenorphine-sustained release) (ZooPharm). The shaved dorsal area was swabbed with applications of povidone-iodine and alcohol. The graft bed was created midway laterally between the dorsal and ventral sides of the mouse by pinching skin with forceps followed by skin excision utilizing a sterile 10 mm diameter biopsy skin punch. The graft was then placed down on the graft bed and covered with an adhesive bandage that was secured with two sterile surgical staples to the skin. Aseptic technique was practiced during the entire procedure. After 5 days, the bandages and staples were removed and monitoring ensued.

TABLE 2
Experimental dosing and treatment protocol for groups of mice

| | Recipient | | Donor | Donor | |
|---|---|---|---|---|---|
| Group | Strain | n | Strain | Tissue | mAb Treatment |
| A | II2rg^{hu/hu} | 10 | BALB/cJ | Tail skin | No mAb |
| B | II2rg^{hu/hu} | 10 | BALB/cJ | Tail skin | REGN1945 (Isotype) |
| C | II2rg^{hu/hu} | 10 | BALB/cJ | Tail skin | REGN7257 |
| | | | | | (anti-hIL2RG) |

Monitoring of skin graft rejection: Monitoring of the skin grafts included the following criteria: (1) Skin grafts that failed to vascularize properly were considered technical failures and excluded from analysis. These grafts will display scabbing and contraction several hours from bandage removal. (2) “Scabbing” and contraction of the graft at later times was used as indicators of graft rejection. The complete rejection timepoint is recorded as the first day where 100% of the graft tissue was necrotic. Rejection onset was recorded as the first day where there were signs of rejection (i.e., redness). Significance was determined by Log-rank (Mantel-Cox) test with Bonferroni correction (adjusted p value 0.00555, K=9).

Detection of donor specific antibodies by flow cytometry: Blood was sampled at the day 56 post-transplant timepoint to assess formation of donor-specific antibodies. CT26.WT (ATCC® CRL-2638™) cells were cultured in tissue culture flasks to 80% confluent. Cells were washed with 1×DPBS and dissociated with TrypLE Express reagent (Gibco) by incubating at room temperature for 5 minutes and washing flask with complete RPMI 1640 media. Cells were then centrifuged (500 g, 10 minutes), and resuspended at 5 million cells/ml with 1×DPBS with 1:50 dilution of 4 μg/mL of Fc block (Tonbo) for 15 minutes at room temperature. The suspension was plated at 250,000 cells/well (50 μL) in a 384 well V-bottom plate.

50 μL of serially diluted sample serum from transplanted mice and from non-engrafted wild type VG mouse (C57BL/6NTac (75%)/129S6SvEvTac (25%)) and wild type BALB/cJ mouse obtained from The Jackson Laboratory was added to its respective well and incubated at 37° C. for 45 minutes. Following 2 washes with MACS buffer (500 g, 4 minutes), the cells were resuspended in 50 μL of LIVE/DEAD™ Fixable Blue Dead Cell Stain Kit (Invitrogen) diluted 1:500 in 1×DPBS at 50 μL total volume per well and incubated at room temperature for 15 minutes. After centrifugation at 500 g for 4 minutes, the supernatant was discarded, and the cells were resuspended in 25 μL of Fc Block (Tonbo) and incubated at 4° C. for 15 minutes. 25 μL of 2× antibody cocktail (Table 2) was then added and incubated at 4° C. for 25 minutes. Cells were washed in MACS buffer following centrifugation (500 g, 4 minutes) by adding 100 μL of MACS™ buffer to each well. Cells were fixed by resuspending cells in 100 μL of Cytofix™ Fixation Buffer (BD) diluted 1:4 in 1×DPBS and incubated at 4° C. for 15 minutes. The samples were then resuspended in MACS buffer after centrifuging and discarding the fixative. Cells were acquired on a BD Fortessa X-20. Acquired events were analyzed with FlowJo (BD). MFIs were derived from cells that were doublet discriminated (FSC-H, FSC-A) and then Live/Dead dye negative. Results plotted were median fluorescent intensity values at the 1/512 dilution of sample serum.

### Results:

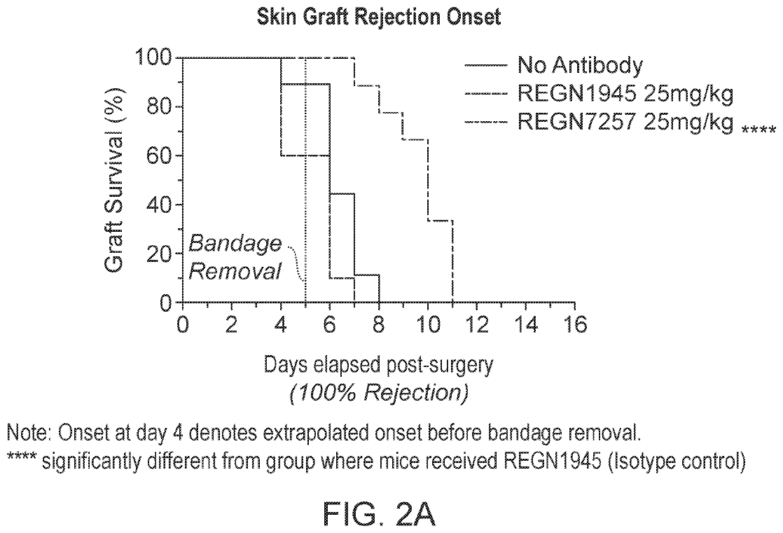

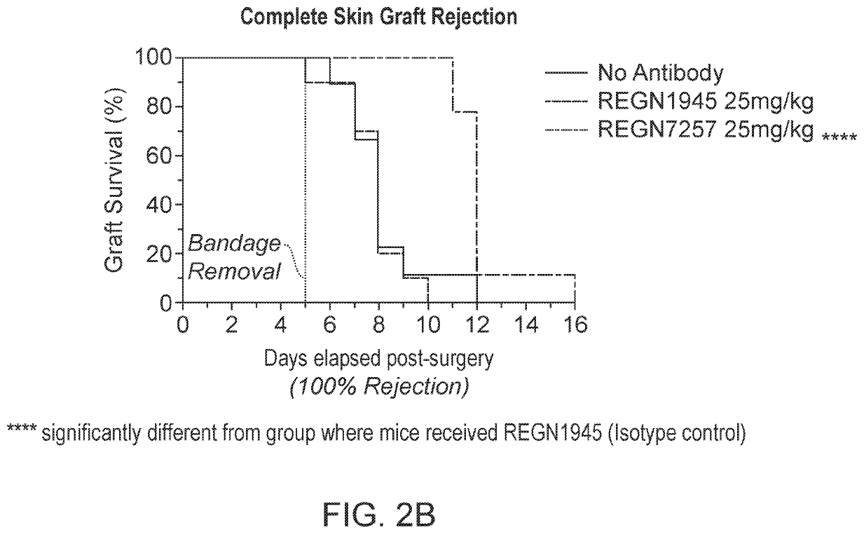

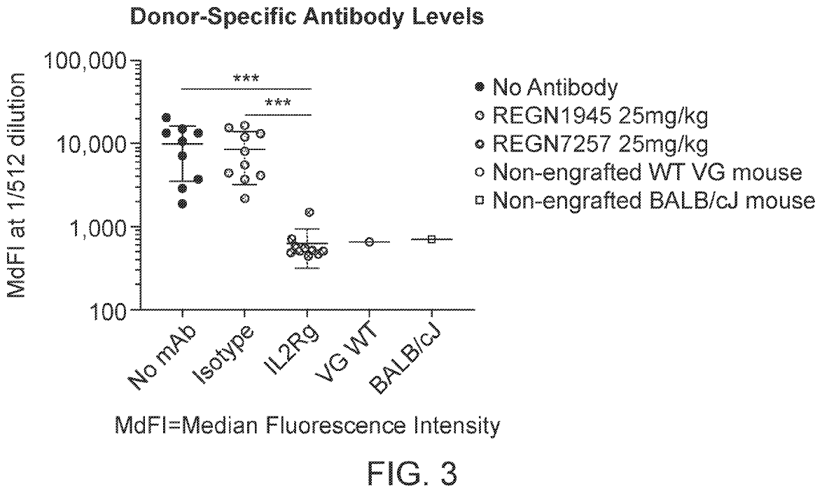

In a skin transplant model (BALB/cJ to VG mice), REGN7257 (IL2RG Ab) treatment delayed onset of skin graft rejection (FIG. 2A) and improved overall skin graft survival (FIG. 2B). REGN7257 treatment also prevented generation of donor-specific antibodies in this transplant model (FIG. 3).

### Example 2: Combination Therapy with Anti-IL-2Rγ and CTLA4-Ig Improves Graft Survival in Skin Transplant Model

An in vivo skin graft rejection model was used to assess the blocking activity of the IL-2Rγ antibody REGN7257 in combination with CTLA4-Ig (ORENCIA® (abatacept)).

### Experimental Procedure:

BALB/cJ mice obtained from The Jackson Laboratory (Bar Harbor, ME) were used as skin graft donors, and MHC mismatched Velocigene® (VG) background mice (C57BL/6NTac (75%)/129S6SvEvTac (25%)) from the Regeneron Velocigene® breeding colony that were genetically modified to replace the endogenous IL2RG ectodomain with the corresponding human sequences were used as recipients. The skin graft was obtained from the tail of the donor mice. The skin was the peeled off using forceps and punched with a 10 mm diameter biopsy punch.

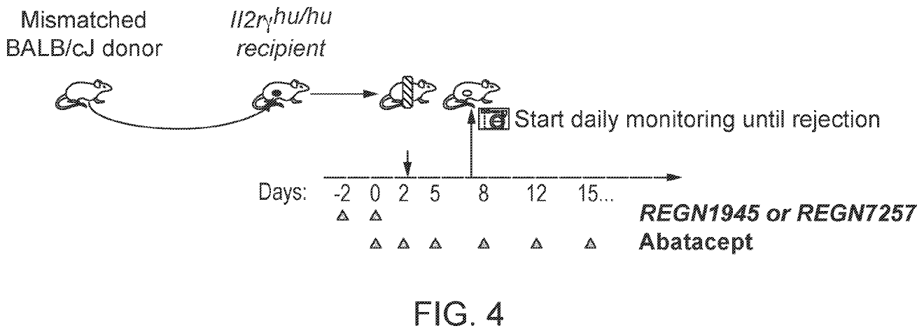

As shown in Table 3 below and in FIG. 4, VG mice (humanized for IL2RG), used as graft recipients, were administered no treatment (negative control); or a treatment regimen as follows:
2 doses of an IgG4 isotype control (REGN1945) or REGN7257 subcutaneously at 25 mg/kg, one dose 2 days prior and one dose the day of the skin transplant (“induction mAb treatment”); and
an IgG1 isotype control (REGN1932) or abatacept subcutaneously at 25 mg/kg at a frequency of 2 times per week starting the day of the transplant, and continuing until rejection (“maintenance mAb treatment”).

TABLE 3
Experimental dosing and treatment protocol for groups of mice

| | Recipient | | Donor | Donor | | |
|---|---|---|---|---|---|---|
| Group | Strain | n | Strain | Tissue | Induction Treatment | Maintenance Treatment |
| A | Il2rg^{hu/hu} | 5 | BALB/cJ | Tail skin | No mAb | No mAb |
| B | Il2rg^{hu/hu} | 9 | BALB/cJ | Tail skin | REGN1945 (IgG4 Isotype) | Abatacept |
| C | Il2rg^{hu/hu} | 10 | BALB/cJ | Tail skin | REGN7257 (anti-hIL2RG) | REGN1932 (IgG1 Isotype) |
| D | Il2rg^{hu/hu} | 9 | BALB/cJ | Tail skin | REGN1945 (IgG4 Isotype) | REGN1932 (IgG1 Isotype) |
| E | Il2rg^{hu/hu} | 10 | BALB/cJ | Tail skin | REGN7257 (anti-hIL2RG) | Abatacept |

Recipients with the surgical site shaved were anesthetized by isoflurane via a nose cone and administered an analgesic (buprenorphine-sustained release) (ZooPharm). The shaved dorsal area was swabbed with applications of povidone-iodine and alcohol. The graft bed was created midway laterally between the dorsal and ventral sides of the mouse by pinching skin with forceps followed by skin excision utilizing a sterile 10 mm diameter biopsy skin punch. The graft was then placed down on the graft bed and covered with an adhesive bandage that was secured with two sterile surgical staples to the skin. Aseptic technique was practiced during the entire procedure. After 5 days, the bandages and staples were removed and monitoring ensued.

Monitoring of skin graft rejection: Monitoring of the skin grafts included the following criteria: (1) Skin grafts that failed to vascularize properly were considered technical failures and excluded from analysis. These grafts will display scabbing and contraction several hours from bandage removal. (2) “Scabbing” and contraction of the graft at later times was used as indicators of graft rejection. The complete rejection timepoint is recorded as the first day where 100% of the graft tissue was necrotic. Significance was determined by Log-rank (Mantel-Cox) test with Bonferroni correction (α=0.005, k=10).

### Results:

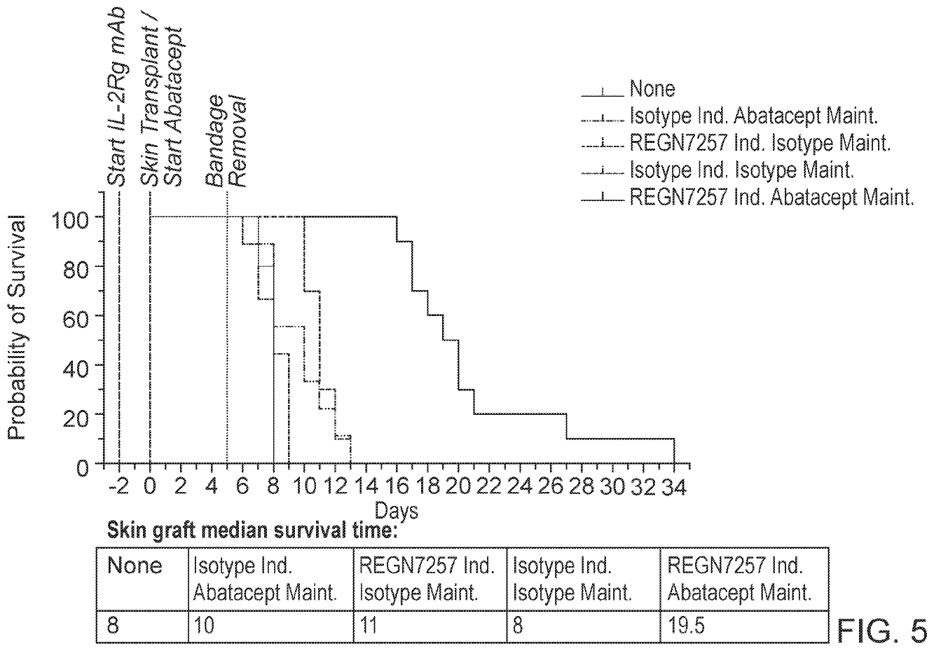

In a skin transplant model (BALB/cJ to VG mice), REGN7257 (IL2RG Ab) and abatacept combination treatment showed a statistically significant increase in skin graft median survival time (i.e., time to complete skin graft rejection) compared to all other groups (p=0.0011 versus “no mAb” group; p=0.0001 versus “isotype control”, p=0.0148 versus “REGN7257 only”, and p=0.0004 versus “abatacept only” groups). Median survival time [MST] for induction therapy with REGN7257 followed by maintenance immunosuppressing using abatacept was 19.5 days, compared to 11 days for REGN7257 induction only or 10 days for abatacept maintenance only. See FIG. 5.

### Example 3: Triple Combination Therapy with Anti-IL-2Rγ, CTLA4-Ig, and Anti-CD40L Improves Graft Survival in Skin Transplant Model

An in vivo skin graft rejection model was used to assess the blocking activity of the IL-2Rγ antibody REGN7257 in combination with CTLA4-Ig (ORENCIA® (abatacept)) and anti-CD40L.

### Assay #1 Experimental Procedure:

BALB/cJ mice obtained from The Jackson Laboratory (Bar Harbor, ME) were used as skin graft donors, and MHC mismatched Velocigene® (VG) background mice (C57BL/6NTac (75%)/129S6SvEvTac (25%)) from the Regeneron Velocigene® breeding colony that were genetically modified to replace the endogenous IL2RG ectodomain with the corresponding human sequences were used as recipients. The skin graft was obtained from the tail of the donor mice. The skin was the peeled off using forceps and punched with a 10 mm diameter biopsy punch.

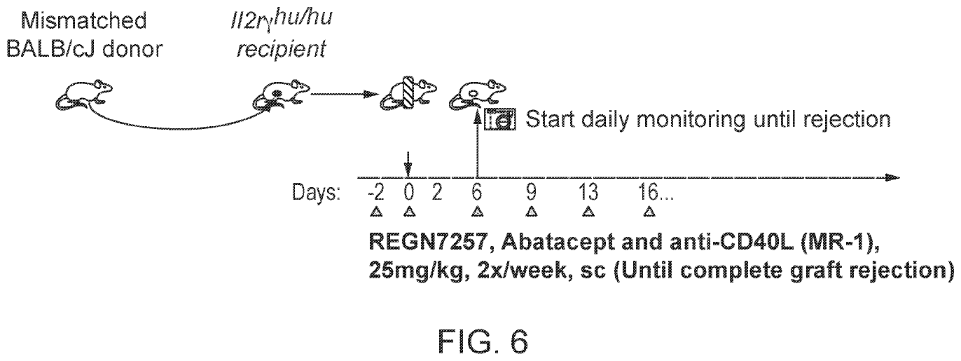

As shown in Table 4 below and in FIG. 6, VG mice (humanized for IL2RG), used as graft recipients, were administered no treatment, an isotype control (REGN1945), abatacept, anti-CD40L (MR-1; BioXCell #BE0017-1), REGN7257, or combination treatments subcutaneously at doses 25 mg/kg at a frequency of 2 times per week starting 2 days prior to transplant, and continuing until rejection. Recipients with the surgical site shaved were anesthetized by isoflurane via a nose cone and administered an analgesic (buprenorphine-sustained release) (ZooPharm). The shaved dorsal area was swabbed with applications of povidone-iodine and alcohol. The graft bed was created midway laterally between the dorsal and ventral sides of the mouse by pinching skin with forceps followed by skin excision utilizing a sterile 10 mm diameter biopsy skin punch. The graft was then placed down on the graft bed and covered with an adhesive bandage that was secured with two sterile surgical staples to the skin. Aseptic technique was practiced during the entire procedure. After 5 days, the bandages and staples were removed and monitoring ensued.

TABLE 4
Experimental dosing and treatment protocol for groups of mice

| | Recipient | | Donor | Donor | |
|---|---|---|---|---|---|
| Group | Strain | n | Strain | Tissue | Maintenance Treatment |
| A | II2rg^{hu/hu} | 5 | BALB/cJ | Tail skin | None |
| B | II2rg^{hu/hu} | 8 | BALB/cJ | Tail skin | IgG4 Isotype (REGN1945) 25 mg/kg |
| C | II2rg^{hu/hu} | 8 | BALB/cJ | Tail skin | Abatacept 25 mg/kg |
| | | | | | IL-2Rg mAb (REGN7257) 25 mg/kg |
| D | II2rg^{hu/hu} | 8 | BALB/cJ | Tail skin | Anti-CD40L (MR-1) 25 mg/kg |
| E | II2rg^{hu/hu} | 8 | BALB/cJ | Tail skin | Abatacept 25 mg/kg |
| | | | | | Anti-CD40L (MR-1) 25 mg/kg |
| F | II2rg^{hu/hu} | 8 | BALB/cJ | Tail skin | IL-2Rg mAb (REGN7257) 25 mg/kg |
| | | | | | Abatacept 25 mg/kg |
| | | | | | Anti-CD40L (MR-1) 25 mg/kg |

Monitoring of skin graft rejection: Monitoring of the skin grafts included the following criteria: (1) Skin grafts that failed to vascularize properly were considered technical failures and excluded from analysis. These grafts will display scabbing and contraction several hours from bandage removal. (2) “Scabbing” and contraction of the graft at later times was used as indicators of graft rejection. The complete rejection timepoint is recorded as the first day where 100% of the graft tissue was necrotic. Significance was determined by Log-rank (Mantel-Cox) test with Bonferroni correction (α=0.00357, k=14).

### Assay #1 Results:

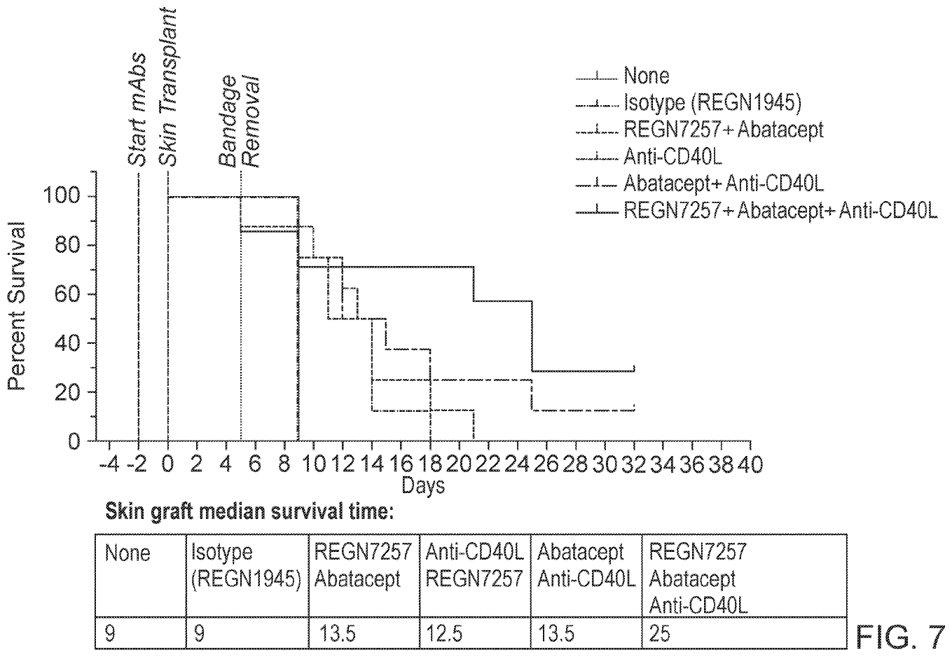

In a skin transplant model (BALB/cJ to VG mice), REGN7257 (IL2RG Ab), abatacept, and anti-CD40L combination treatment prolonged skin graft median survival time (i.e., time to complete skin graft rejection) compared to other treatment groups. See FIG. 7.

### Assay #2 Experimental Procedure:

This assay was performed according to the procedures described for Assay #1, except that one cohort of animals (Group D) was administered anti-CD40L antibody in combination with REGN7257. See Table 5.

TABLE 5
Experimental dosing and treatment protocol for groups of mice

| | Recipient | | Donor | Donor | |
|---|---|---|---|---|---|
| Group | Strain | n | Strain | Tissue | Maintenance Treatment |
| A | II2rg^{hu/hu} | 5 | BALB/cJ | Tail skin | None |
| B | II2rg^{hu/hu} | 8 | BALB/cJ | Tail skin | IgG4 Isotype (REGN1945) 25 mg/kg |
| C | II2rg^{hu/hu} | 8 | BALB/cJ | Tail skin | Abatacept 25 mg/kg |
| | | | | | IL-2Rg mAb (REGN7257) 25 mg/kg |
| D | II2rg^{hu/hu} | 8 | BALB/cJ | Tail skin | IL-2Rg mAb (REGN7257) 25 mg/kg |
| | | | | | Anti-CD40L (MR-1) 25 mg/kg |
| E | II2rg^{hu/hu} | 8 | BALB/cJ | Tail skin | Abatacept 25 mg/kg |
| | | | | | Anti-CD40L (MR-1) 25 mg/kg |
| F | II2rg^{hu/hu} | 8 | BALB/cJ | Tail skin | IL-2Rg mAb (REGN7257) 25 mg/kg |
| | | | | | Abatacept 25 mg/kg |
| | | | | | Anti-CD40L (MR-1) 25 mg/kg |

### Assay #2 Results:

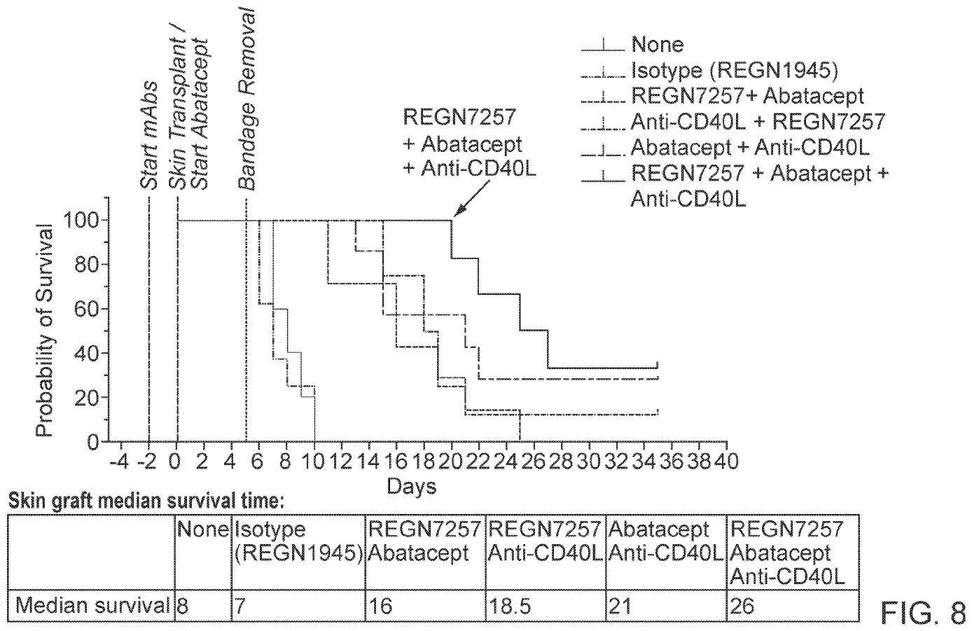

As shown in FIG. 8, maintenance immunosuppression combining blocking of γc cytokine signaling together with blocking of both CD28 and CD40L costimulatory signals prolonged skin graft survival better than any combination therapies of only 2 pathway blockers (MST=26 days for REGN7257+abatacept+anti-CD40L, versus MST of 16, 18.5, or 21 days for REGN7257+abatacept, REGN7257+anti-CD40L, or abatacept+anti-CD40L, respectively).

### Example 4: Combination Therapy with Anti-IL-2Rγ and MMF Improves Graft Survival in Skin Transplant Model

An in vivo skin graft rejection model was used to assess the blocking activity of the IL-2Rγ antibody REGN7257 in combination with the antiproliferative agent mycophenolate mofetil (MMF).

### Experimental Procedure:

BALB/cJ mice obtained from The Jackson Laboratory (Bar Harbor, ME) were used as skin graft donors, and MHC mismatched Velocigene® (VG) background mice (C57BL/6NTac (75%)/129S6SvEvTac (25%)) from the Regeneron Velocigene® breeding colony that were genetically modified to replace the endogenous IL2RG ectodomain with the corresponding human sequences were used as recipients. The skin graft was obtained from the tail of the donor mice. The skin was the peeled off using forceps and punched with a 10 mm diameter biopsy punch.

As shown in Table 6 below, VG mice (humanized for IL2RG), used as graft recipients, were administered no treatment, an isotype control (REGN1945), MMF, REGN7257, or a combination of REGN7257 and MMF. REGN7257 was administered subcutaneously at doses of 25 mg/kg at a frequency of 2 times per week starting 2 days prior to transplant, and continuing until rejection. MMF was administered intraperitoneally daily at a dose 100 mg/kg starting on the day of transplant, and continuing until rejection. Recipients with the surgical site shaved were anesthetized by isoflurane via a nose cone and administered an analgesic (buprenorphine-sustained release) (ZooPharm). The shaved dorsal area was swabbed with applications of povidone-iodine and alcohol. The graft bed was created midway laterally between the dorsal and ventral sides of the mouse by pinching skin with forceps followed by skin excision utilizing a sterile 10 mm diameter biopsy skin punch. The graft was then placed down on the graft bed and covered with an adhesive bandage that was secured with two sterile surgical staples to the skin. Aseptic technique was practiced during the entire procedure. After 5 days, the bandages and staples were removed and monitoring ensued. Monitoring of skin graft rejection was performed as described in Examples 1-3 above.

TABLE 6
Experimental dosing and treatment protocol for groups of mice

| | Recipient | | Donor | |
|---|---|---|---|---|
| Group | Strain | n | Strain | Maintenance Treatment |
| A | II2rg^{hu/hu} | 5 | BALB/cJ | None |
| B | II2rg^{hu/hu} | 5 | BALB/cJ | Dextrose |
| | | | | IgG4 Isotype (REGN1945) |
| | | | | 25 mg/kg |
| C | II2rg^{hu/hu} | 10 | BALB/cJ | MMF 100 mg/kg |
| D | II2rg^{hu/hu} | 10 | BALB/cJ | IL-2Rg mAb (REGN7257) |
| | | | | 25 mg/kg |
| E | II2rg^{hu/hu} | 10 | BALB/cJ | MMF 100 mg/kg |
| | | | | IL-2Rg mAb (REGN7257) |
| | | | | 25 mg/kg |

### Results:

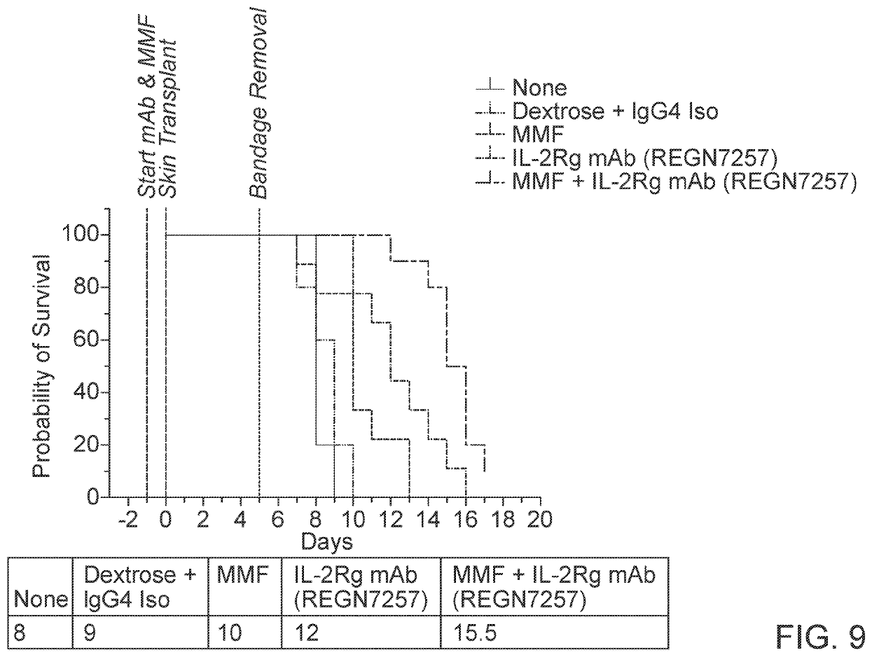

In treatment combination experiments, monotherapy treatment with the antiproliferative agent MMF was not as efficient as REGN7257 at delaying skin graft rejection (Median Survival Time [MST]_{MMF}=10 versus MST_{IL2RG}=12). Simultaneous blockade of cell proliferation and yc cytokine signaling conferred an advantage over monotherapies (MST_{MMF+IL2RG}=15.5). See FIG. 9.

The present disclosure is not to be limited in scope by the specific embodiments described herein. Indeed, various modifications of the disclosure in addition to those described herein will become apparent to those skilled in the art from the foregoing description and the accompanying figures. Such modifications are intended to fall within the scope of the appended claims. The disclosures of all patents and non-patent literature cited herein are expressly incorporated in their entirety by reference.

What is claimed is:

1. A method of improving organ or tissue transplant survival, the method comprising:
administering to a subject at least one dose of an antibody that specifically binds to interleukin 2 receptor gamma chain (IL-2Rγ), or an antigen-binding fragment thereof, prior to an organ or tissue transplant in the subject; and
administering to the subject one or more doses of a CD28 signaling inhibitor.

2. The method of claim 1, wherein the IL-2Rγ antibody or antigen-binding fragment thereof comprises three HCDRs (HCDR1, HCDR2 and HCDR3) and three LCDRs (LCDR1, LCDR2 and LCDR3), wherein the HCDR1 comprises the amino acid sequence of SEQ ID NO:2, the HCDR2 comprises the amino acid sequence of SEQ ID NO:3, the HCDR3 comprises the amino acid sequence of SEQ ID NO:4, the LCDR1 comprises the amino acid sequence of SEQ ID NO:6, the LCDR2 comprises the amino acid sequence AAS, and the LCDR3 comprises the amino acid sequence of SEQ ID NO:8.

3. The method of claim 1 or 2, wherein the IL-2Rγ antibody or antigen-binding fragment thereof comprises a heavy chain variable region (HCVR) comprising the amino acid sequence of SEQ ID NO:1 and a light chain variable region (LCVR) comprising the amino acid sequence of SEQ ID NO:5.

4. The method of any one of claims 1 to 3, wherein the IL-2Rγ antibody or antigen-binding fragment thereof comprises a heavy chain comprising the amino acid sequence of SEQ ID NO:9 and a light chain comprising the amino acid sequence of SEQ ID NO:10.

5. The method of any one of claims 1 to 4, wherein the method comprises administering to the subject two or more doses of the IL-2Rγ antibody or antigen-binding fragment thereof prior to the organ or tissue transplant.

6. The method of any one of claims 1 to 4, wherein the method comprises administering to the subject one or more doses of the IL-2Rγ antibody or antigen-binding fragment thereof prior to the organ or tissue transplant and one or more doses of the IL-2Rγ antibody or antigen-binding fragment thereof after the organ or tissue transplant.

7. The method of any one of claims 1 to 6, wherein the IL-2Rγ antibody or antigen-binding fragment thereof is administered to the subject once a week, two times a week, or three times a week.

8. The method of any one of claims 1 to 7, wherein the CD28 signaling inhibitor is a fusion protein comprising an extracellular portion of cytotoxic T lymphocyte-associated protein 4 (CTLA4) and an Fc region of immunoglobulin IgG1 (Ig).

9. The method of claim 8, wherein the CTLA4-Ig fusion protein is abatacept or belatacept.

10. The method of any one of claims 1 to 9, wherein the CD28 signaling inhibitor is administered to the subject starting on the day of the organ or tissue transplant.

11. The method of any one of claims 1 to 9, wherein at least one dose of the CD28 signaling inhibitor is administered to the subject prior to the organ or tissue transplant.

12. The method of any one of claims 1 to 11, wherein the CD28 signaling inhibitor is administered to the subject three times a week, two times a week, once a week, or once every two weeks.

13. The method of any one of claims 1 to 12, wherein the method further comprises administering to the subject a CD40L inhibitor.

14. The method of claim 13, wherein the CD40L inhibitor is a CD40L antibody or an antigen-binding fragment thereof, or a fusion protein that specifically binds to CD40L.

15. The method of claim 13 or 14, wherein the CD40L inhibitor is dapirolizumab pegol, dazodalibep, frexalimab, letolizumab, ruplizumab, tegoprubart, or MR-1.

16. The method of any one of claims 13 to 15, wherein the CD40L inhibitor is administered to the subject once a week, two times a week, or three times a week.

17. The method of any one of claims 1 to 16, wherein:
(a) the CD28 signaling inhibitor is abatacept and the CD40L inhibitor is dapirolizumab pegol, dazodalibep, frexalimab, letolizumab, ruplizumab, tegoprubart, or MR-1; or
(b) the CD28 signaling inhibitor is belatacept and the CD40L inhibitor is dapirolizumab pegol, dazodalibep, frexalimab, letolizumab, ruplizumab, tegoprubart, or MR-1; or
(c) the CD28 signaling inhibitor is abatacept and the CD40L inhibitor is MR-1; or
(d) the CD28 signaling inhibitor is belatacept and the CD40L inhibitor is MR-1.

18. The method of any one of claims 1 to 17, wherein one or more of the IL-2Rγ antibody or antigen-binding fragment thereof, the CD28 signaling inhibitor, and the CD40L inhibitor are administered to the subject starting at least two days prior to the organ or tissue transplant.

19. The method of any one of claims 1 to 18, wherein one or more of the IL-2Rγ antibody or antigen-binding fragment thereof, the CD28 signaling inhibitor, and the CD40L inhibitor are administered to the subject until the onset of transplant rejection.

20. A method for delaying the onset of transplant rejection in a subject, the method comprising:
administering to a subject at least one dose of an antibody that specifically binds to IL-2Rγ, or an antigen-binding fragment thereof, prior to an organ or tissue transplant in the subject; and
administering to the subject one or more doses of a CD28 signaling inhibitor.

21. The method of claim 20, wherein the IL-2Rγ antibody or antigen-binding fragment thereof comprises three HCDRs (HCDR1, HCDR2 and HCDR3) and three LCDRs (LCDR1, LCDR2 and LCDR3), wherein the HCDR1 comprises the amino acid sequence of SEQ ID NO:2, the HCDR2 comprises the amino acid sequence of SEQ ID NO:3, the HCDR3 comprises the amino acid sequence of SEQ ID NO:4, the LCDR1 comprises the amino acid sequence of SEQ ID NO: 6, the LCDR2 comprises the amino acid sequence AAS, and the LCDR3 comprises the amino acid sequence of SEQ ID NO:8.

22. The method of claim 20 or 21, wherein the IL-2Rγ antibody or antigen-binding fragment thereof comprises an HCVR comprising the amino acid sequence of SEQ ID NO: 1 and an LCVR comprising the amino acid sequence of SEQ ID NO:5.

23. The method of any one of claims 20 to 22, wherein the IL-2Rγ antibody or antigen-binding fragment thereof comprises a heavy chain comprising the amino acid sequence of SEQ ID NO: 9 and a light chain comprising the amino acid sequence of SEQ ID NO:10.

24. The method of any one of claims 20 to 23, wherein the method comprises administering to the subject two or more doses of the IL-2Rγ antibody or antigen-binding fragment thereof prior to the organ or tissue transplant.

25. The method of any one of claims 20 to 23, wherein the method comprises administering to the subject one or more doses of the IL-2Rγ antibody or antigen-binding fragment thereof prior to the organ or tissue transplant and one or more doses of the IL-2Rγ antibody or antigen-binding fragment thereof after the organ or tissue transplant.

26. The method of any one of claims 20 to 25, wherein the IL-2Rγ antibody or antigen-binding fragment thereof is administered to the subject once a week, two times a week, or three times a week.

27. The method of any one of claims 20 to 26, wherein the CD28 signaling inhibitor is a CTLA4-Ig fusion protein.

28. The method of claim 27, wherein the CTLA4-Ig fusion protein is abatacept or belatacept.

29. The method of any one of claims 20 to 28, wherein the CD28 signaling inhibitor is administered to the subject starting on the day of the organ or tissue transplant.

30. The method of any one of claims 20 to 28, wherein at least one dose of the CD28 signaling inhibitor is administered to the subject prior to the organ or tissue transplant

31. The method of any one of claims 20 to 30, wherein the CD28 signaling inhibitor is administered to the subject once a week or two times a week.

32. The method of any one of claims 20 to 31, wherein the method further comprises administering to the subject a CD40L inhibitor.

33. The method of claim 32, wherein the CD40L inhibitor is a CD40L antibody or an antigen-binding fragment thereof, or a fusion protein that specifically binds to CD40L.

34. The method of claim 32 or 33, wherein the CD40L inhibitor is dapirolizumab pegol, dazodalibep, frexalimab, letolizumab, ruplizumab, tegoprubart, or MR-1.

35. The method of any one of claims 32 to 45, wherein the CD40L inhibitor is administered to the subject once a week, two times a week, or three times a week.

36. The method of any one of claims 20 to 35, wherein:
(a) the CD28 signaling inhibitor is abatacept and the CD40L inhibitor is dapirolizumab pegol, dazodalibep, frexalimab, letolizumab, ruplizumab, tegoprubart, or MR-1; or
(b) the CD28 signaling inhibitor is belatacept and the CD40L inhibitor is dapirolizumab pegol, dazodalibep, frexalimab, letolizumab, ruplizumab, tegoprubart, or MR-1; or
(c) the CD28 signaling inhibitor is abatacept and the CD40L inhibitor is MR-1; or
(d) the CD28 signaling inhibitor is belatacept and the CD40L inhibitor is MR-1.

37. The method of any one of claims 20 to 36, wherein one or more of the IL-2Rγ antibody or antigen-binding fragment thereof, the CD28 signaling inhibitor, and the CD40L inhibitor are administered to the subject starting at least two days prior to the organ or tissue transplant.

38. The method of any one of claims 20 to 37, wherein one or more of the IL-2Rγ antibody or antigen-binding fragment thereof, the CD28 signaling inhibitor, and the CD40L inhibitor are administered to the subject until the onset of transplant rejection.

39. A method of improving organ or tissue transplant survival or delaying the onset of transplant rejection, the method comprising:
administering to a subject at least one dose of an antibody that specifically binds to IL-2Rγ, or an antigen-binding fragment thereof, prior to an organ or tissue transplant in the subject; wherein the subject is taking or has been prescribed a CD28 signaling inhibitor.

40. The method of claim 39, wherein the CD28 signaling inhibitor is a CTLA4-Ig fusion protein.

41. The method of claim 40, wherein the CTLA4-Ig fusion protein is abatacept or belatacept.

42. The method of any one of claims 39 to 41, wherein the subject is taking the CD28 signaling inhibitor at least one day before the organ or tissue transplant.

43. The method of any one of claims 39 to 41, wherein the subject is taking the CD28 signaling inhibitor starting on the day of the organ or tissue transplant.

44. The method of any one of claims 39 to 43, wherein the IL-2Rγ antibody or antigen-binding fragment thereof comprises three HCDRs (HCDR1, HCDR2 and HCDR3) and three LCDRs (LCDR1, LCDR2 and LCDR3), wherein the HCDR1 comprises the amino acid sequence of SEQ ID NO:2, the HCDR2 comprises the amino acid sequence of SEQ ID NO:3, the HCDR3 comprises the amino acid sequence of SEQ ID NO:4, the LCDR1 comprises the amino acid sequence of SEQ ID NO:6, the LCDR2 comprises the amino acid sequence AAS, and the LCDR3 comprises the amino acid sequence of SEQ ID NO:8.

45. The method of any one of claims 39 to 44, wherein the IL-2Rγ antibody or antigen-binding fragment thereof comprises an HCVR comprising the amino acid sequence of SEQ ID NO: 1 and an LCVR comprising the amino acid sequence of SEQ ID NO:5.

46. The method of any one of claims 39 to 45, wherein the IL-2Rγ antibody or antigen-binding fragment thereof comprises a heavy chain comprising the amino acid sequence of SEQ ID NO: 9 and a light chain comprising the amino acid sequence of SEQ ID NO:10.

47. The method of any one of claims 39 to 46, wherein the method comprises administering to the subject:
two or more doses of the IL-2Rγ antibody or antigen-binding fragment thereof prior to the organ or tissue transplant; or
one or more doses of the IL-2Rγ antibody or antigen-binding fragment thereof prior to the organ or tissue transplant and one or more doses of the IL-2Rγ antibody or antigen-binding fragment thereof after the organ or tissue transplant.

48. The method of any one of claims 39 to 47, wherein the IL-2Rγ antibody or antigen-binding fragment thereof is administered to the subject once a week, two times a week, or three times a week.

49. A combination for use in improving organ or tissue transplant survival or for use in delaying the onset of transplant rejection in a subject, the combination comprising:
an antibody that specifically binds to IL-2Rγ or an antigen-binding fragment thereof; and
a CD28 signaling inhibitor.

50. The combination of claim 49, wherein the IL-2Rγ antibody or antigen-binding fragment thereof comprises three HCDRs (HCDR1, HCDR2 and HCDR3) and three LCDRs (LCDR1, LCDR2 and LCDR3), wherein the HCDR1 comprises the amino acid sequence of SEQ ID NO:2, the HCDR2 comprises the amino acid sequence of SEQ ID NO:3, the HCDR3 comprises the amino acid sequence of SEQ ID NO:4, the LCDR1 comprises the amino acid sequence of SEQ ID NO: 6, the LCDR2 comprises the amino acid sequence AAS, and the LCDR3 comprises the amino acid sequence of SEQ ID NO:8.

51. The combination of claim 50, wherein the IL-2Rγ antibody or antigen-binding fragment thereof comprises an HCVR comprising the amino acid sequence of SEQ ID NO:1 and an LCVR comprising the amino acid sequence of SEQ ID NO:5.

52. The combination of any one of claims 49 to 51, wherein the IL-2Rγ antibody or antigen-binding fragment thereof comprises a heavy chain comprising the amino acid sequence of SEQ ID NO: 9 and a light chain comprising the amino acid sequence of SEQ ID NO: 10.

53. The combination of any one of claims 49 to 52, wherein the CD28 signaling inhibitor is a CTLA4-Ig fusion protein.

54. The combination of claim 53, wherein the CTLA4-Ig fusion protein is abatacept or belatacept.

55. The combination of any one of claims 49 to 54, further comprising a CD40L inhibitor, wherein the CD40L inhibitor is a CD40L antibody or an antigen-binding fragment thereof, or a fusion protein that specifically binds to CD40L.

56. The combination of claim 55, wherein the CD40L inhibitor is dapirolizumab pegol, dazodalibep, frexalimab, letolizumab, ruplizumab, tegoprubart, or MR-1.

57. The method of any one of claims 1 to 48 or the combination of any one of claims 49 to 56, wherein the organ or tissue transplant is a bone transplant, corneal transplant, heart transplant, heart valve transplant, kidney transplant, ligament transplant, liver transplant, lung transplant, pancreas transplant, skin transplant, tendon transplant, trachea transplant, or vascular tissue transplant.

58. A method of improving organ or tissue transplant survival or delaying the onset of transplant rejection in a subject, the method comprising:
administering to a subject at least one dose of an antibody that specifically binds to interleukin 2 receptor gamma chain (IL-2Rγ), or an antigen-binding fragment thereof, prior to an organ or tissue transplant in the subject;
administering to the subject one or more doses of an antiproliferative agent.

59. The method of claim 58, wherein the IL-2Rγ antibody or antigen-binding fragment thereof comprises three HCDRs (HCDR1, HCDR2 and HCDR3) and three LCDRs (LCDR1, LCDR2 and LCDR3), wherein the HCDR1 comprises the amino acid sequence of SEQ ID NO:2, the HCDR2 comprises the amino acid sequence of SEQ ID NO:3, the HCDR3 comprises the amino acid sequence of SEQ ID NO:4, the LCDR1 comprises the amino acid sequence of SEQ ID NO: 6, the LCDR2 comprises the amino acid sequence AAS, and the LCDR3 comprises the amino acid sequence of SEQ ID NO:8.

60. The method of claim 58 or 59, wherein the IL-2Rγ antibody or antigen-binding fragment thereof comprises a heavy chain variable region (HCVR) comprising the amino acid sequence of SEQ ID NO: 1 and a light chain variable region (LCVR) comprising the amino acid sequence of SEQ ID NO:5.

61. The method of any one of claims 58 to 60, wherein the IL-2Rγ antibody or antigen-binding fragment thereof comprises a heavy chain comprising the amino acid sequence of SEQ ID NO: 9 and a light chain comprising the amino acid sequence of SEQ ID NO:10.

62. The method of any one of claims 58 to 61, wherein the method comprises administering to the subject two or more doses of the IL-2Rγ antibody or antigen-binding fragment thereof prior to the organ or tissue transplant.

63. The method of any one of claims 58 to 61, wherein the method comprises administering to the subject one or more doses of the IL-2Rγ antibody or antigen-binding fragment thereof prior to the organ or tissue transplant and one or more doses of the IL-2Rγ antibody or antigen-binding fragment thereof after the organ or tissue transplant.

64. The method of any one of claims 58 to 63, wherein the IL-2Rγ antibody or antigen-binding fragment thereof is administered to the subject once a week, two times a week, or three times a week.

65. The method of any one of claims 58 to 64, wherein the antiproliferative agent is mycophenolate mofetil (MMF).

66. The method of any one of claims 58 to 65, wherein the antiproliferative agent is administered to the subject starting on the day of the organ or tissue transplant.

67. The method of any one of claims 58 to 65, wherein at least one dose of the antiproliferative agent is administered to the subject prior to the organ or tissue transplant.

68. The method of any one of claims 58 to 67, wherein the antiproliferative agent is administered to the subject daily.

69. The method of any one of claims 58 to 68, wherein one or both of the IL-2Rγ antibody or antigen-binding fragment thereof and the antiproliferative agent are administered to the subject until the onset of transplant rejection.

70. A method of improving organ or tissue transplant survival or delaying the onset of transplant rejection, the method comprising:
administering to a subject at least one dose of an antibody that specifically binds to IL-2Rγ, or an antigen-binding fragment thereof, prior to an organ or tissue transplant in the subject; wherein the subject is taking or has been prescribed an antiproliferative agent.

71. The method of claim 70, wherein the antiproliferative agent is MMF.

72. The method of claim 70 or 71, wherein the subject is taking the antiproliferative agent at least one day before the organ or tissue transplant.

73. The method of any one of claims 70 to 72, wherein the subject is taking the antiproliferative agent starting on the day of the organ or tissue transplant.

74. The method of any one of claims 70 to 73, wherein the IL-2Rγ antibody or antigen-binding fragment thereof comprises three HCDRs (HCDR1, HCDR2 and HCDR3) and three LCDRs (LCDR1, LCDR2 and LCDR3), wherein the HCDR1 comprises the amino acid sequence of SEQ ID NO:2, the HCDR2 comprises the amino acid sequence of SEQ ID NO:3, the HCDR3 comprises the amino acid sequence of SEQ ID NO:4, the LCDR1 comprises the amino acid sequence of SEQ ID NO:6, the LCDR2 comprises the amino acid sequence AAS, and the LCDR3 comprises the amino acid sequence of SEQ ID NO:8.

75. The method of any one of claims 70 to 74, wherein the IL-2Rγ antibody or antigen-binding fragment thereof comprises an HCVR comprising the amino acid sequence of SEQ ID NO: 1 and an LCVR comprising the amino acid sequence of SEQ ID NO:5.

76. The method of any one of claims 70 to 75, wherein the IL-2Rγ antibody or antigen-binding fragment thereof comprises a heavy chain comprising the amino acid sequence of SEQ ID NO: 9 and a light chain comprising the amino acid sequence of SEQ ID NO:10.

77. The method of any one of claims 70 to 76, wherein the method comprises administering to the subject:
two or more doses of the IL-2Rγ antibody or antigen-binding fragment thereof prior to the organ or tissue transplant; or
one or more doses of the IL-2Rγ antibody or antigen-binding fragment thereof prior to the organ or tissue transplant and one or more doses of the IL-2Rγ antibody or antigen-binding fragment thereof after the organ or tissue transplant.

78. The method of any one of claims 70 to 77, wherein the IL-2Rγ antibody or antigen-binding fragment thereof is administered to the subject once a week, two times a week, or three times a week.

79. A combination for use in improving organ or tissue transplant survival or for use in delaying the onset of transplant rejection in a subject, the combination comprising:
an antibody that specifically binds to IL-2Rγ or an antigen-binding fragment thereof; and
an antiproliferative agent.

80. The combination of claim 79, wherein the IL-2Rγ antibody or antigen-binding fragment thereof comprises three HCDRs (HCDR1, HCDR2 and HCDR3) and three LCDRs (LCDR1, LCDR2 and LCDR3), wherein the HCDR1 comprises the amino acid sequence of SEQ ID NO:2, the HCDR2 comprises the amino acid sequence of SEQ ID NO:3, the HCDR3 comprises the amino acid sequence of SEQ ID NO:4, the LCDR1 comprises the amino acid sequence of SEQ ID NO: 6, the LCDR2 comprises the amino acid sequence AAS, and the LCDR3 comprises the amino acid sequence of SEQ ID NO:8.

81. The combination of claim 80, wherein the IL-2Rγ antibody or antigen-binding fragment thereof comprises an HCVR comprising the amino acid sequence of SEQ ID NO:1 and an LCVR comprising the amino acid sequence of SEQ ID NO:5.

82. The combination of any one of claims 79 to 81, wherein the IL-2Rγ antibody or antigen-binding fragment thereof comprises a heavy chain comprising the amino acid sequence of SEQ ID NO: 9 and a light chain comprising the amino acid sequence of SEQ ID NO:10.

83. The combination of any one of claims 79 to 82, wherein the antiproliferative agent is MMF.


SEQUENCE LISTING (10 sequences)

SEQ ID NO: 1 - protein, 121 aa, synthetic construct
EVQLVESGGGLVQPGGSLRLSCAASGFIFSSYEMHWVRQAPGKGLEWISYISSSGTTIYYADSVKGRFTISRDNAKNSLYLHMNSLRAEDTAVYYCTRARITGTFDVFDIWGQGTMVTVSS

SEQ ID NO: 2 - protein, 8 aa, synthetic construct
GFIFSSYE

SEQ ID NO: 3 - protein, 8 aa, synthetic construct
ISSSGTTI

SEQ ID NO: 4 - protein, 14 aa, synthetic construct
TRARITGTFDVFDI

SEQ ID NO: 5 - protein, 107 aa, synthetic construct
DIQMTQSPSSLSASVGDRVTITCRASQSISSYLNWYQQKPGKAPKLLIFAASNLQSGVPSRFSGSRSGTDFTLTISSLQPEDFATYYCQQNYNIPYTFGQGTKLEIK

SEQ ID NO: 6 - protein, 6 aa, synthetic construct
QSISSY

SEQ ID NO: 7 - skipped

SEQ ID NO: 8 - protein, 9 aa, synthetic construct
QQNYNIPYT

SEQ ID NO: 9 - protein, 448 aa, synthetic construct
EVQLVESGGGLVQPGGSLRLSCAASGFIFSSYEMHWVRQAPGKGLEWISYISSSGTTIYYADSVKGRFTISRDNAKNSLYLHMNSLRAEDTAVYYCTRARITGTFDVFDIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 214 aa, synthetic construct
DIQMTQSPSSLSASVGDRVTITCRASQSISSYLNWYQQKPGKAPKLLIFAASNLQSGVPSRFSGSRSGTDFTLTISSLQPEDFATYYCQQNYNIPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC